(19) United States
(12) Patent Application Publication
(10) Pub. No.: US 2010/0292178 A1
(43) Pub. Date: Nov. 18, 2010

(54) Method of treating non-insulin dependent diabetes mellitus and related complications

(76) Inventor: Jeffrey Young, Rosemead, CA (US)

Correspondence Address: DAVID AND RAYMOND PATENT FIRM, 108 N. YNEZ AVE., SUITE 128, MONTEREY PARK, CA, 91754, US

(21) Appl. No.: 12/799,750

(22) Filed: Apr. 30, 2010

Related U.S. Application Data
(63) Continuation-in-part of application No. 10/771,711, filed on Feb. 3, 2004, now abandoned.
(60) Provisional application No. 60/488,893, filed on Jul. 21, 2003.

(51) Int. Cl. A61K 31/7048; A61P 3/10

(52) U.S. Cl. 514/27

(57) ABSTRACT

A method of restoring insulin beta cells in a living object to lower a plasma sugar level comprising the step of providing a composition of berberine and catalpol as the two major active ingredients of said composition. The composition is preferably include oleanolic acid and taurine as active ingredients. The composition is pre-made into a single dosage unit. The single dosage contains active ingredients within a range between 1 to 300 mg/kg/dl for a single intake and is in a form of packet powders, solution in vials, ampoules or pre-filled syringes, capsule, tablet, draught in water, syrup and/or cachets. The berberine is obtained from natural herbs Berberis, Chelidonium, Stephniz, Coptis, Phellodendron, and/or Ziziphus, the catalpol is obtained from natural herbs Rehmannia, Verbascum, Panulownia, Glubularia and/or Adonis. The oleanolic acid is obtained from natural herbs Olea, Swertia, Astrantia, Lonicera and/or Beta.

### CROSS REFERENCE OF RELATED APPLICATION

This is a Continuation-in-part application that claims the benefits of priority under 35 U.S.C. §119 to a non-provisional application, application Ser. No. 10/771,711, filed Feb. 3, 2004, which claimed the benefits of priority of a provisional application, application No. 60/488,893, filed Jul. 21, 2003.

### BACKGROUND OF THE PRESENT INVENTION

1. Field of Invention

The present invention relates to a method of treating non-insulin dependent diabetes mellitus (NDDM) and related complications, and more particularly to a method of treating NDDM with a composition derived from natural herbs comprising berberine and catalpol wherein the berberine and the catalpol are active ingredients for treating NDDM.

2. Description of Related Arts

Diabetes mellitus is a syndrome, characterized by abnormal insulin level which may be resulted from absolute or insufficient insulin production or decreased sensitivity to insulin. Since insulin is a key hormone for glucogenesis, the abnormal insulin level will generally induce an elevated plasma glucose level which poses different significant effects. Some of the possible symptoms of diabetes mellitus are frequent urination, excess thirst, extreme hunger, weight loss, high blood pressure and glucose urine. In some cases, usually found in severe cases, ketoacidosis may be developed as a result of extraordinary high blood glucose level, which may even cause death

Non-insulin dependent diabetes mellitus, commonly known as NDDM or Type II diabetes, is used to categorize the group of diabetics having at least a certain level of insulin production ability while failing to utilize or ineffectively response to the insulin. Normally, insulin treatment is not required for NDDM and typical triggers for NDDM are obesity, poor diet and lifestyle. Type II diabetes is typically characterized by obesity, hyperglycemia, abnormal insulin production, hyperinsulinemia and insulin resistance.

Complications of diabetes mellitus include a number of disabling conditions such as neuropathy, retinopathy and vasculopathy. Because of high blood glucose level, cells and hence tissues damage will be induced in some particular parts of the body when excessive glucose is converted into fructose and sorbitol which is capable of causing swelling and impairing biological cell functions, and having reaction with proteins and nucleic acids such that premature ‘cell aging’ is promoted. Generally speaking, type II diabetics will have a shortened life expectancies compared with the healthy population. The most important health hazard of diabetes mellitus is generally caused by the elevated blood glucose level. Therefore, one of the key treatments for diabetes mellitus is a good control of blood plasma glucose level so as to manage the disease.

Orally effective anti-hyperglycemic agents are often used to reduce plasma glucose levels and to reduce damage to nervous, retinal, renal and vascular systems. Different mechanisms are employed including inhibition of fatty acid oxidation, inhibition of alpha-glycosidase, antagonism of alpha-2-receptors and inhibition of gluconeogenesis. At present, there are four common classes of agent, namely biguanides, sulfonylureas, thiazolidinediones and arylalkyl compounds. Representative drugs for the four classes are pheniformin, tolbutamide, ciglitazone, and N-arylalky-N-hydroxy urea and 2(arylalkyl)-[1,2,4-oxadiazolidine-3,5-diones] respectively. Ciglitazone can suppress the symptoms of diabetes hyperglycemia, hypertriglyceridemia and hyperinsulinemia [Diabetes 32, 804-10 (1983)]. The hypoglycemic properties of these drugs in ob/ob mice are discussed by Goldstein et al. in J. Med. Chem. 36, 2238-2240 (1993). In summary, animal experiments showed that sulfonylurea is effective on normal and type II diabetic animals but is ineffective on tetroxide alloxan diabetic animals while biguanide is effective on tetroxide alloxan diabetes animals but ineffective on naturally diabetic animals.

Some synthetic compounds are developed to block or slow down the conversion of glucose to fructose and sorbitol in order to relieve the peripheral symptoms of diabetes. These compounds are capable of inhibiting the enzyme aldosoreductase to prevent glucose from forming sorbitol and thus reducing the damage caused by cellular edema (Annual Reports in Medicine Chemistry 19, 169-177, 1984). In short-term clinical tests, these compounds were used to antagonize diabetic neuropathies (Lancet II, 758-762, 1983; New England J. Medicine 316, 599-606, 1987). However, these synthetic compounds have side effects and may be incompatible with long-term use ordinarily expected for diabetic patients.

Insulin may be used for type II diabetic patients to control blood glucose level. However, insulin must generally be administered by injection because oral administration of insulin is ineffective owing to the fact that insulin will be easily broken down by digestive enzymes. Though research for means other than injection has been conducted, injection is still the most effective means of controlling the dosage of insulin. Routine insulin injections are of course uncomfortable or even painful as well as expensive.

Controlling the plasma glucose level by anti-hyperglycemic agents alone is probably not the most effective treatment for type II diabetes and complications especially at advanced stages of the disease. Some studies investigating pathological changes of diabetic blood vessels by the World Health Organization show that the risk of suffering coronary heart disease amongst diabetic patients was correlated with an increased total cholesterol level and elevated low-density lipoprotein (LDL) concentration. It was shown that a reduction in LDL concentration in blood could minimize the risk of coronary heart disease. In addition, diabetic patients also exhibit various degrees of changes in hematological properties such as a considerable increase of whole blood viscosity, fibrinogen content and red blood cells count or concentration in diabetic patients compared with the correspondent values of normal individuals. As a result of the poor blood circulation, microcirculation in tissues is severely retarded and oxygen shortage in tissues inevitably occurs. These pathological changes cause the development of diabetic blood vessels. Furthermore, since the elevation of whole blood viscosity and blood plasma viscosity is correlated to an elevated level of cholesterol and apolipoprotein, a more suitable treatment for diabetic patients having diabetic blood vessels should include medicine for maintaining or restoring the impaired vascular system.

Other diabetic vascular complications are chiefly related to artheroma caused by cholesterol metabolism disorder. Abnormal cholesterol metabolism is mainly characterized by elevated total cholesterol, triglycerides, low-density lipoprotein concentration and apolipoprotein-B, and reduced apoliproprotein-A1 and high-density lipoprotein (HDL) concentration. Clinical studies indicate that an elevation of cholesterol level is correlated to the higher likelihood of artheroma eruption.

The pathological changes of diabetic blood vessels are closely related to the changes in cholesterol, lipoprotein and apolipoprotein metabolisms. An increase in platelet's viscosity and platelet aggregation degree is also often seen in diabetic patients, which in turn leads to abnormal blood coagulation that is one of the major causes of artheroma. Elevated blood viscosity is attributed to the reduced concentration of high-density lipoprotein and the elevated total cholesterol concentration. Compared with non-diabetic individuals, the ages at which artheroma erupts amongst the diabetic patients are younger wherein the diabetic patients are distributed in wider symptoms. The abnormal blood viscosity leads to an increased heart beat rate, brain and kidney diseases, and pathological changes of peripheral blood vessels.

Therefore, a strategic approach to type II diabetes and peripheral complications should include measures for effectively addressing cholesterol metabolism disorders and abnormal hematological problems.

As a result, a method of treating type II diabetes comprising the steps for reducing the plasma glucose level as well as posing beneficially influence to the vascular system without observable adverse side effects for long term use is vital for the diabetic patients. Such method will be expected to bring a compound effect to the diabetic patients. Natural herbs are used gradually for treating diseases. Diabetes mellitus, in the aspect of the traditional Chinese medicine, is one of the imbalance signal generated for giving a warming signal. Elevated blood glucose generally will not cause chronic and acute hazards but rather will lead to the development of long-term complication when this kind of imbalance is ignored.

Synthetic medicine inherited the disadvantage of unknown or undiscovered chemical reaction to our body which is harmful to health. On the other hand, natural herbs are found in nature which generally do not have unknown or undesirable side effects on our body under normal consumption and so natural herbs are gaining importance in treating diseases. Therefore, this will be a great breakthrough in medical science if it is possible to restore the balance or to cure a disease with the use of naturally occurred herbs instead of artificial synthetic chemicals or medicines.

At present, a variety of treatments are used for type II diabetes mellitus, such as insulin injection, administration of synthetic antihyperglycemic agent, and exercise planning. There exists no method of treatment which is capable of utilization of natural herb ingredients. Furthermore, there exists no method of identification of active ingredients of natural herbs, too. Therefore, if it is possible to identify and utilize any active ingredients in nature herbs for treating type II diabetes mellitus, methods of treatments for diabetic patients may be improved significantly.

### SUMMARY OF THE PRESENT INVENTION

The invention is advantageous in that it provides a method of treating NDDM and related complications with a composition derived from natural herbs comprising berberine and catalpol wherein the berberine and the catalpol are active ingredients for treating NDDM.

Another advantage of the present invention is to provide a method of treating diabetes mellitus and related complications with a composition comprising berberine and catalpol so as to increase the quantity of insulin level and the sensitivity with respect to the insulin.

Another advantage of the present invention is to provide a method of using berberine or a composition containing berberine and catalpol to treat type II diabetes of human beings, diabetic mice, domestic rabbits, and other living subjects.

Another advantage of the present invention is to provide a method of treating diabetes mellitus with a composition comprising berberine, catalpol and oleanolic acid.

Another advantage of the present invention is to provide a method of using berberine or a composition containing berberine, catalpol and oleanolic acid to treat type II diabetes of human beings, diabetic mice, domestic rabbits, and other living subjects.

Another advantage of the present invention is to provide a method of using berberine or a composition containing berberine and catalpol to raise the insulin level of type II diabetic patients, normal kk mice, and spontaneously diabetic mice.

Another advantage of the present invention is to provide a method of using berberine or a composition containing berberine and catalpol for increasing the number of beta cells to restore the functions of the beta cells.

Another advantage of the present invention is to provide a method of using berberine or a composition containing berberine and catalpol which is capable of having combined characteristics of both sulfonylurea and biguanides for reducing plasma glucose level of normal mice, spontaneously diabetic kk mice, and tetroxide alloxan mice.

Another advantage of the present invention is to provide a method of using berberine or a composition containing berberine wherein the berberine or the composition containing berberine, in a predetermined therapeutically effective amount, is capable of neat administration or is preferably to be administered with a pharmaceutical carrier such as a solid carrier, a liquid carrier including water carrier, and a gas carrier.

Another advantage of the present invention is to provide a method of using berberine or a composition containing berberine so as to reducing blood cholesterol level.

Another advantage of the present invention is to provide a method of using berberine or a composition containing berberine to reduce blood cholesterol levels of living subjects such as mice which had been fed with high cholesterol emulsions such that the method is capable of inhibiting domestic rabbit's platelet aggregation induced by adenosine diphosphate (ADP).

Another advantage of the present invention is to provide a method of using berberine or a composition containing berberine to reduce blood cholesterol level such that the method is useful in improving abnormal blood coagulation property of diabetic patients and hence the method may be used to treat cholesterol metabolism disorder, abnormal platelet aggregation and blood coagulation problems of diabetes patients.

Another advantage of the present invention is to provide a method of treating diabetes mellitus with berberine or a composition comprising berberine to lower plasma concentrations of total cholesterol, triglyceride, low-density lipoprotein, and apolipoprotein B, and to raise plasma concentrations of high-density lipoprotein and apolipoprotein A1 in diabetic patients such that the method is effective in lowering whole blood viscosity, blood plasma viscosity, fibrinogen and red cells content of diabetic patients.

Another advantage of the present invention is to provide a method of treating diabetes mellitus with a composition comprising berberine and catalpol wherein the berberine and the catalpol are in a relative ratio in the range from 1:20 to 20:1 by weight.

Another advantage of the present invention is to provide a method of treating diabetes mellitus with a composition comprising berberine, catalpol and oleanolic acid such that the composition is effective in raising insulin level, increasing the number of insulin beta cells and/or restoring their functions, and reducing plasma glucose level, promoting a transformation of plasma lipoprotein while eliminating cholesterolemia and promoting cholesterol metabolism, increasing concentration of high-density lipoprotein and apolipoprotein A1 in blood plasma, inhibiting platelet aggregation and improving blood coagulation property to improving blood circulation and microcirculation of tissues and curing diabetic arteries and other microcirculatory diseases, and retarding the spreading of local infection due to its antibiotic activity.

Another advantage of the present invention is to provide a method of using a pharmaceutical composition comprising berberine, catalpol and oleanolic acid such that the composition is effective in raising insulin level of type II diabetic living subjects such as human beings or spontaneously diabetic or induced-diabetic mice, increasing the number of insulin beta cells and/or restoring their functions of living subjects, and reducing plasma glucose level of diabetic living subjects such as tetroxide alloxan diabetic mice, promoting a transformation of plasma lipoprotein while eliminating cholesterolemia and promoting cholesterol metabolism, increasing concentration of high-density lipoprotein and apolipoprotein A1 in blood plasma, inhibiting platelet aggregation and improving blood coagulation property of living subjects to improving blood circulation and microcirculation of tissues and curing diabetic arteries and other microcirculatory diseases, and retarding the spreading of local infection due to its antibiotic activity.

Another advantage of the present invention is to provide a method of treating diabetes mellitus with composition comprising berberine and catalpol to reduce plasma glucose level wherein the dose of berberine is equal to one-tenth of its deadly dose and the composition is in the forms of solid, liquid, powder, gas or other physical forms used for therapeutic drugs.

Another advantage of the invention is to provide a method of treating diabetes mellitus with a composition comprising berberine and catalpol wherein the quantity of berberine is one-tenth of its lethal dose whereby the composition is capable of reducing blood glucose level.

Another advantage of the invention is to provide a method of treating diabetes mellitus using compositions obtained from natural sources including edible plants such that when the dosage is not excessive and disproportional to the weight of an individual, the composition is safe to the individual who does not have special genetic defects or special physiological problems and that no side effects is induced.

Additional advantages and features of the invention will become apparent from the description which follows, and may be realized by means of the instrumentalities and combinations particular point out in the appended claims.

According to the present invention, the foregoing and other objects and advantages are attained by a process of treating diabetes mellitus and related complications comprising steps of providing a composition comprising a predetermined amount of berberine and a predetermined amount of catalpol, wherein the composition may further comprise a predetermined amount of oleanolic acid.

In accordance with another aspect of the invention, the present invention comprises a composition of treating non-insulin dependent diabetes and related complications, comprising a berberine as a first active ingredient and a catalpol as a second active ingredient, wherein the composition may further comprise an oleanolic acid as a third active ingredient.

Preferably, the composition is prepared into a predetermined form for administration that contains 1 to 300 mg/kg/dl of the berberine, and preferably that contains 5 to 150 mg/kg/dl of the berberine. The form for administration includes draught in water, syrup, caches, tablet and solution.

Alternately, the composition is prepared into a predetermined form for administration that contains 1 to 300 mg/kg/dl of the first and second active ingredients, or the first, second and third active ingredients.

In accordance with another aspect of the invention, the present invention comprises a method of treating of a living object with a disease selected from the group consisting of insulin independent diabetes, cholesterol elevation, and hyperglycemia, wherein the method comprises a step of: administrating to the living object a pharmaceutical composition containing an active compound selected from the group consisting of a barbering and salts of barbering in a therapeutically effective dose in a pharmaceutically acceptable carrier to the living object.

In particular, the dose of berberine is in a range of 1-300 mg·kg/dl, and preferably in a range of 5-100 mg/kg/dl.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1A to 1C illustrates the chemical structures of three interchangeable forms of berberine of the present invention.

FIG. 2 illustrates the chemical structure of catalpol of the present invention.

FIG. 3 is the chemical structure of oleanolic acid of the present invention.

FIG. 4 is a flow diagram showing the extraction and separation process for obtaining berberine of the present invention.

FIG. 5 is a flow diagram showing the extraction and separation process for obtaining catalpol of the present invention.

FIG. 6 is a flow diagram showing the extraction and separation process for obtaining oleanolic acid of the present invention.

FIG. 7 is a chromatogram obtained by High Performance Liquid Chromatography for identification of berberine.

FIG. 8 is bar chart showing the effect of berberine and catalpol on lowering the serum glucose of normal mice.

FIG. 9 is a chart showing the serum glucose level of normal mice in a six-hour period after a predetermined amount of berberine and catalpol have been administered.

FIG. 10 is a chart showing the serum glucose level of normal mice in a three-hour period in response to intrapertoned administration of a predetermined amount of glucose solution wherein the normal mice was treated with a predetermined amount of berberine and catalpol.

FIG. 11 is a chart showing the effect of berberine and catalpol on glucose tolerance of the normal mice.

FIG. 12 is a table showing effects of berberine and catalpol on plasma sugar levels of mice.

FIG. 13 is a table showing effects of berberine and catalpol on insulin beta cell count.

FIG. 14 is a table showing effects of berberine and catalpol on plasma glucose level of normal mice.

FIG. 15 is a table showing effects of berberine and catalpol on plasma glucose to level elevation caused by adrenaline.

FIG. 16 is a table showing effects of berberine and catalpol on plasma glucose level of tetraoxide alloxan diabetic mice.

FIG. 17 is a table showing effects of berberine and catalpol on platelet aggregation of rabbits in vitro.

FIG. 18 is a table showing changes of plasma sugar, cholesterol, lipoprotein and apolipoprotein of different predetermined groups.

FIG. 19 is a table showing hematological properties of the treatment group and the control group before and after treatment in the Example 13.

### DETAILED DESCRIPTION OF THE PREFERRED EMBODIMENT

Berberine is one of the active ingredients found in nature herbs normally for antibiotic functions. Berberine typically has inhibition effect on dysentery bacillus, staphylococcus, and streptococcus and hence is used for treatment in enteritis, dysentery and inflammation. Furthermore, berberine is also commonly used for cancer treatment. However, no study has shown that berberine is capable of affecting glucose cycle and treating diabetes mellitus.

Catalpol and Oleanolic acid are other active ingredients found in nature herbs. Catalpol is generally used for lowering blood pressure while oleanolic acid has antibiotic effects. Still, no studies have shown that catalpol and oleanolic acid are correlated to diabetes mellitus treatments.

On the other hands, there is a still difficulty in identification of these useful phytochemicals and the use of these chemicals is somewhat limiting. Therefore, if these chemicals can be identified and extracted, their uses will be explored dramatically.

Berberine and catalpol are commonly existed in many plants, however, their therapeutic effects on type II diabetes and related complications are not known. Furthermore, these active ingredients can be taken orally, and so is a good alternative to insulin injection.

Essentially all materials disclosed hereinafter were translated from publications in foreign language. None of any single chemical or physical characteristic such as formula weight, stereo structure, reactivity, existing sources in nature, maximum absorption wavelength, absorption profiles, exciting wavelengths, optical property, emission wavelengths, extraction and separation solvent and conditions, melting points, existing color, crystal type, affinity to solvents, and solubility, is intended as a criterion for identifying the compounds which are the active ingredients of the present invention. These characteristics are used collectively to describe and identify these compounds. A limited number of studies have been done to understand the structures and properties of those compounds.

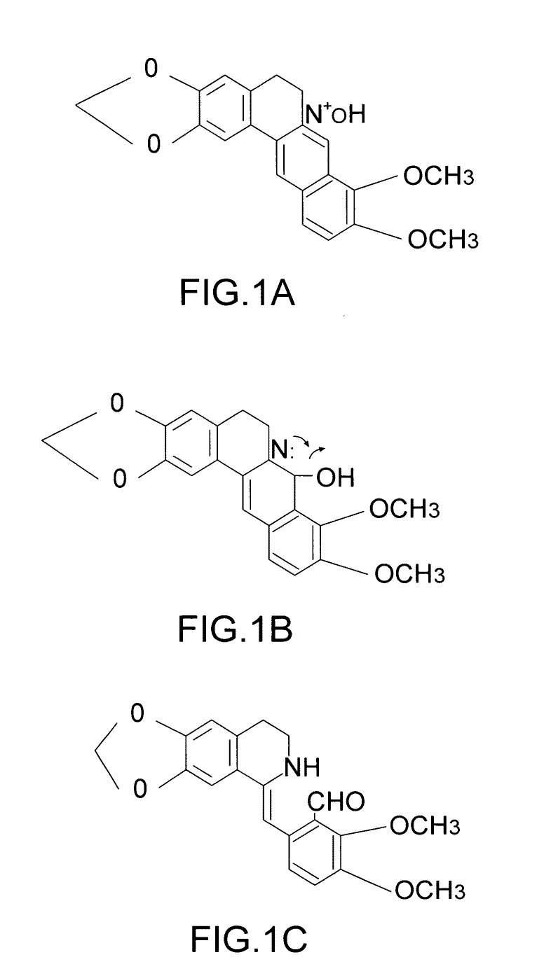

Referring to FIGS. 1 to 3 of the drawings, the chemical structures of berberine, catalpol and oleanolic acid are disclosed. Berberine, catalpol, and oleanolic acid used in the present invention are found in a variety of plants. For example, berberine are found in the rhizomes, stems, wood portion, and root epidermal layer of Berberis Thunbergii, in the root portion of B. Julianae Schneid, in the root portion of B. Wilsonae Hemsl, in the leaves of Mahonia Japonica (Thunb) DC, in the seeds of Chelidonium Majus L., in the roots of Stephnia Cepharantha hayata, in the rhizomes of Coptis Chinensis Franch, in the epidermal layer and fruit of Phellodendron Amurense Rupr. And in the leaves of Ziziphus Jujuba Mill. Catalpol are found in the rhizomes of Rehmannia glutinosa (Gaerth) Libosch, in the whole plant of Verbascum thapsus L., in the wood portion of Panulownia tomentosa (thund) Steud, in the flower and leaves of Verbascum Lychnitis L., in the exposed portion of the plant above soil, and in the whole plant of Adonis Szechuanensis Franch. Oleanolic acid is commonly found in leaves of Olea Europaca L., in the fruit of Ligustrum lucisum Ait., in the whole plant of Swertia Mileensis T. N. He et W. L. Shi, in the leaves and roots of Astrantia major L., in Lonicera Nigra and in Beta Unlgaris L.

Referring to FIGS. 1 to 3 of the drawings, a molecular formula and a molecular weight of berberine are C₂₀H₁₈NO₄ and 336.37 respectively; a molecular formula and a molecular weight of catalpol are C₁₅H₂₂O₁₀ and 362.34 respectively; and a molecular formula and a molecular weight of oleanolic acid are C₃₀H₄₈O₃ and 456.71 respectively.

Referring to FIGS. 1A to 1C of the drawings, the three interchangeable forms of berberine are disclosed. The three forms of berberine are the quaternary ammonium form which is red brown in color in solution, the alcohol form which is yellow in color, and the aldehyde form which is yellow in color. The quaternary ammonium form of berberine salt, which is a free berberine as a tertiary hydrate, is a yellow or orange crystal. The berberine salt is highly soluble in water to form a strong alkaline solution.

Free berberine is capable of slowly dissolving in cold water (1:20), easily dissolving in hot water, hot alcohol, and cold alcohol (1:100), and slightly dissolving in phenyl, chloroform and acetone solution. Berberine, obtained from crystallization in water or diluted alcoholic solution, is a needle-like yellow crystal having 5.5 water molecules. Under drying at 100° C., 2.5 water molecules are remained. Under heating, berberine changes its color to an intensive darkish color at 110° C. and starts to decompose at 160° C. Hydroxylated berberine is a needle-like yellow crystal (ethyl) having a melting point of 145° C., chlorinated berberine hydrated with two water molecules is a yellow crystal which starts to decompose at around 220° C. and forms a red brown berberrubine which is saturated at 285° C. Berberine salt is slightly soluble in cold water, easily dissolved in hot water, and almost insoluble in alcohol, chloroform and ether. Berberine salt exhibits different solubility in water under room temperature, such as hydrochloric berberine (1:500), hydriodic berberine (1:2130), citric berberine (1:125), berberine phosphate salt (1:15), berberine sulfate (1:30), and berberine sulfite (1:100). A berberine salt formed with large molecular organic acid has poor solubility in water, for example, berberine analyte is obtained from berberine which forms sparely soluble salt or molecular complex with glycerate and rhubarb tannic acid.

Quaternary ammonium, alcohol and aldehyde are the three interchangeable forms of berberine wherein the quaternary berberine is the most stable structure. All kinds of berberine salts belong to the quaternary berberine. Adding a predetermined amount of barium hydroxide into an aqueous solution of sulfuric acid and berberine will produce a free red brown quaternary ammonium berberine which is highly basic, highly soluble in water but insoluble in ether. If the amount of the barium hydroxide is added in excess, a precipitate of alcohol berberine will be formed. If berberine salts are mixed with excessive amount of sodium hydroxide, free berberine which is soluble in ether will be produced. When sodium hydroxide is added into a solution of berberine salt to form an alkaline solution, an addition of a few drops of acetone will result in the formation of yellow crystals of acetone berberine. The melting point, which is within a certain range, indicates that berberine contains the structure of alpha hydroxylamine. This reaction can be used to indicate the existence of berberine. On the other hand, when an acidic solution of berberine is added with reagent such as bleaching powder or calcium hypochlorate, the solution will turn cherry-red, and that this reaction can also be used to indicate the existence of berberine.

Berberine has obvious antibiotic activity which is especially effective in inhibiting dysentery bacillus, staphylococcus, and streptococcus and has been used for clinical treatment for enteritis and dysentery. Other organic alkalis also show obvious anti-inflammatory function.

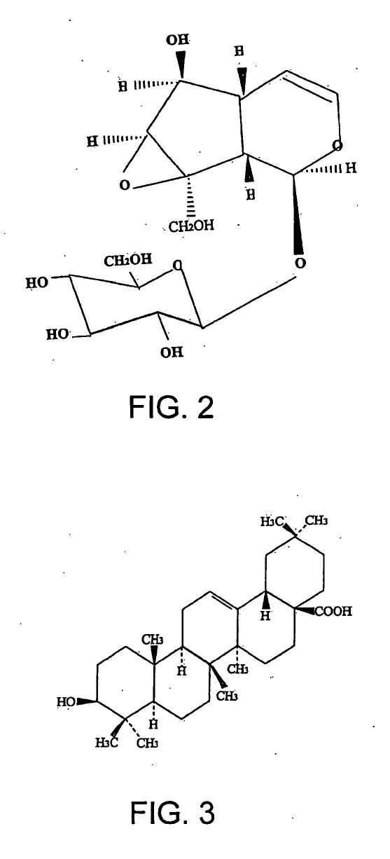

Referring to FIG. 2 of the drawings, catalpol, which is also called methyl iridoid glycoside (a 7,8-cycloether), is normally a colorless and shapeless powder. Its melting point is between 207° C. and 209° C. After hydrolysis, it will become an unstable compound and its color is easily be changed under exposure to environmental conditions. When it is under certain conditions, such as high temperature and exposure to light, it is easily turned to a dark color. It is highly soluble in water and methanol, soluble in ethanol, acetone, and butan-1-ol, but almost insoluble in lipophilic organic solvents such as chloroform, benzene, and petroleum ether solution. It has bitter taste and has active chiral atoms. Its' [α]²³_{D} is about 122° C. in dilute ethanol.

Referring to FIG. 3 of the drawings, oleanolic acid exists as a free compound or as a compound bound to carbohydrates. When it is bound to carbohydrates, it is called triterpene saponin. Pentacyclic triterpenoids are basically terpenoid compounds containing 30 carbon atoms and are commonly found in nature. Most terpenoid are oxygen-containing derivatives and exhibit biological activities. Terpenoid molecules are relatively large and therefore it is difficult to be crystallized. They are often existed in the forms of colorless or cream-like color shapeless powders. The melting point is relatively high and decomposition will take place before melting. Thus, they have no measurable melting points. They are highly soluble in water, hot methanol and ethanol, moderately soluble in water-containing butanol and therefore butanol is often used as the solvent for extraction which can enhance the solubility of other compounds in water.

Catalpol has some common observable properties. One of the properties is froth-forming property. Shaking an aqueous solution of catalpol can produce lasting froth (bubbles). The froth will not disappear even when the solution is heated. This property is attributed to the capability of saponins to reduce the surface tension of the aqueous solution. Another property is that saponin in an aqueous solution is able to damage red blood cells, and this property is related to the hemolysis of living subjects. Accordingly, both intravenous and intra muscular injections of saponins in living subjects are not used. However, if they are taken orally, no hemolysis is observed which is perhaps because the intestinal and digestive system cannot absorb them directly.

Hemolysis is attributed to saponins's ability to bind cholesterol to form water insoluble complex compounds. When saponins in an aqueous solution contact red blood cells, the cholesterol in the wall of the red blood cells binds the saponins to form complex precipitates. This reaction alters the osmotic property of the membrane of the red blood cells and cause the osmotic pressure inside the red blood cells to increase. The collapse of the red blood cells caused by the increased osmotic pressure is the cause of hemolysis.

However, not all saponins exhibit hemolytic effect. In general, hemolytic effect of a single saponins is obvious whereas, that of double saponins, including some of the neutral saponins, is weaker or non-existing.

Optical rotation properties of saponins are useful in determining their chemical structures. Generally, solid saponins and optically active saponaceous components in complex can cause polarized light to rotate left. In addition, the degrees by which they can rotate polarized light appear to be related to the double bonds.

All cholesterol containing C₃-β-OH can bind saponins to form water insoluble compounds. If C₃-β-OH of a steroid is in a form, the steroid cannot react with saponins to for insoluble compounds. As far as saponinfication is concerned, the compound formed from solid saponins and steroids is more stable than the compounds produced from saponaceous triterpernoids and steroids. This saponaceous property can be used in extraction and separation for them.

Precipitates can result from mixing the aqueous solution of saponins with metal salts such as those of lead, barium, and copper. Adding sulfuric ammonium and other neutral salts such as acetic lead to the acidic solution of saponins will also produce precipitates. For a neutral aqueous solution of saponins to form precipitates, it is necessary to add basic salts such as acetic lead or barium hydroxide. This property can be used in extraction and separation for saponins.

Hydrolysis of saponins or the cleavage of the glucosidic bond is important in studying the structure of saponins. There are many methods to break the bonds wherein acid hydrolysis is the most frequently used method. Sometimes, the reaction conditions for acid hydrolysis can cause many saponins undergo transformation. Thus, other methods have also been developed.

Smith decomposition is an extension of the oxidative reaction of excessive salt of iodic acid. It employs a diluted inorganic acid to control the hydrolysis of polyhydric alcohols under room temperature. The hydrolytic condition is mild, allowing many saponins unstable under acidic hydrolytic conditions to form genuine hydrolytic products.

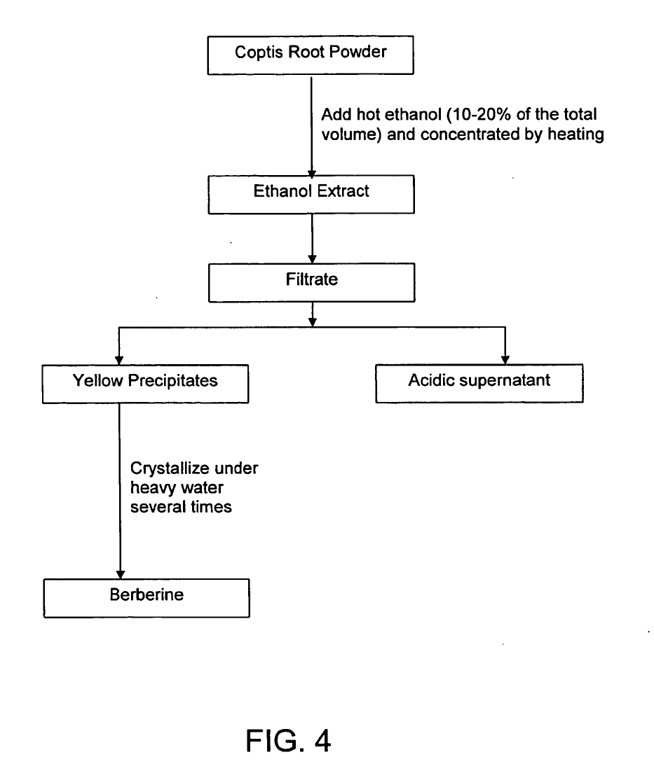

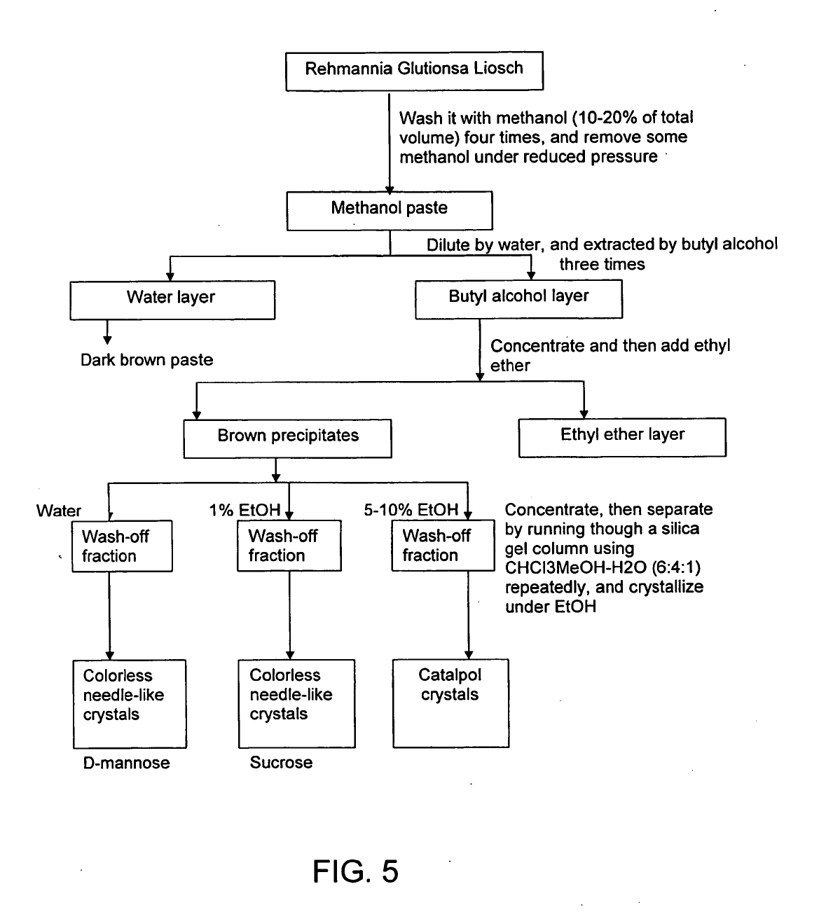

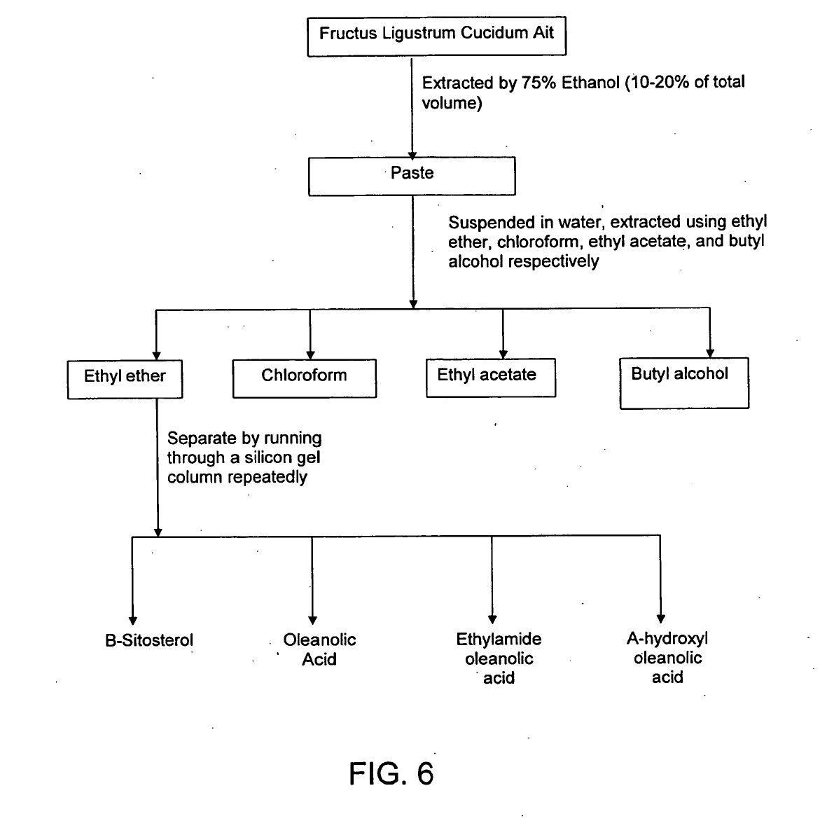

Referring to FIGS. 4 to 6 of the drawings, the extraction and separation processes for obtaining berberine, catalpol, and oleanolic acid are shown respectively. One of the extraction and separation methods of berberine was described by Xiao Zhongyuan (Chinese Herbal Chemistry, Xianghai Science and Technology Publisher, 1987, 103-104) and by Yang Zisheng et al. (Northern Western Medicinal Chemistry, China, 1987, 11(1): 16-17). According to the method, the starting material, Coptis Root Powder, is mixed with warm ethanol which is about 10% to 20% of the total final volume, for several hours and is concentrated under heating or a reduced pressure condition. After the concentrated solution is allowed to equilibrate for about 12 hours, it is then filtered, thereby removing residues and yielding a filtrate. Then, concentrated hydrochloric acid is added to the filtrate to produce precipitates. Two hours later, the acidic supernatant is discarded, and the yellow precipitates are collected. The yellow precipitates are then crystallized several times in heavy water which then give rise to berberine. The yielding percentage is about 8%.

Catalpol can be extracted according to the method described by Liu Wen Ru (Chinese Herbal Chemistry, Xiue Wuan (Scienc Variety) Publisher, P. R. China, 1995, 341). The starting material, such as 10 kg of Rehmannia Glutionsa Libosch, is washed with methanol (10-20% of the final volume) four times and all extracted solution or suspension is collected and combined. The extract is concentrated under reduced pressure to yield a methanol paste which is about 20% of the starting materials by weight (wt/wt). Water is added to dilute the paste to yield a suspension and butanol is added to the suspension to extract soluble materials. The butanol is then separated from water and the water layer is discarded or collected for other purposes. The butanol layer is concentrated under reduced pressure and is added with ether. The ether is separated from butanol and the yellow brown solid in the butanol layer is washed with water, 1% ethanol, and 5-10% ethanol respectively to yield three fractions of solutions. The first two, obtained from water and 1% ethanol, are used for other purposes. The fraction from 5-10% ethanol is concentrated and allowed to passing silicon separation column using running solvent such as chloroform, methanol, and water in the ratio of 6:4:1. A target compound, which is identified by strong saponaceous property, is collected, concentrated by re-running through the column, and crystallized under ethanol, thus yielding catalpol.

Compound oleanolic acid may be extracted according to the method described by Yishuan et al. (Sengyan University Medicinal Journal, P.R. China, 1995, 12(2): 125-126). The starting material, such as 10 kg of Fructus Ligustrum Cucidum Ait, is extracted with ethanol in 10-20% of final volume several times, and the extract is combined and concentrated, yielding about 3000 g of ethanol paste. Water is added to the paste to form a suspension, which is extracted using ethyl ether, chloroform, ethyl ethanoate and butyl alcohol (butan-1-ol) respectively. The ethyl ether extract is collected and is allowed to passing a silicon separation column several times, yielding oleanolic acid in one of the fractions. The other extracts are used for other purposes.

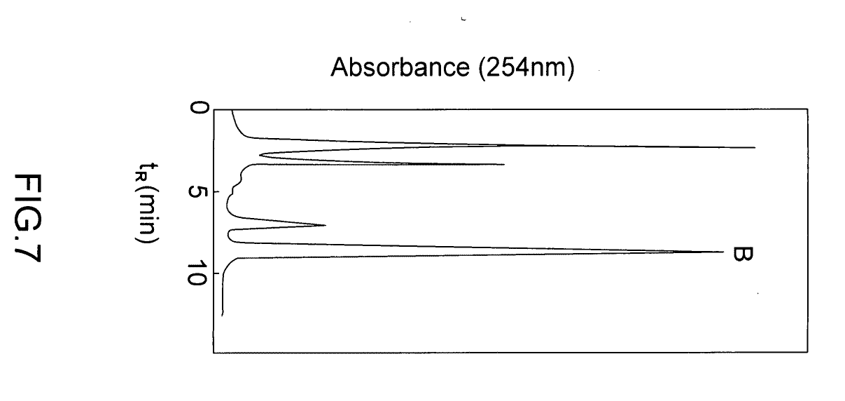

Referring to FIG. 7 of the drawings, berberine is quantified by chromatography under the following conditions: 1000×2.1 mm Zipax SCX column; mobile phase of 0.1M NaClO₄ (CH₃CN and H₂O at ratio of 6:4)-0.2 M HBO₃, and 0.002 M NaClO₄ at pH 8.5; operating pressure at the inlet: 1000 lb/in²; temperature: 45° C.; flow rate: 0.93 ml/min; and detection wavelength: 254 nm.

A standard curve is prepared by adding 2 mg of berberine into 10 ml methanol. 1˜3 μl of sample was taken and injected into the HPLC system. Regression equation between the amount of berberine and the height of the absorption peak at 254 nm is established by using least square method. A typical regression result is: height (mm)=13.806 amount (μg)+0.0025 (r=0.999). A similar regression equation is found between the peak area and the amount.

In a typical sample analysis, 5.0 g f Coptidis Rhizoma (J.P.V. III) is placed in a flask of suitable size. Methanol is used to extract it repeatedly until it is colorless. Methanol is removed under reduced pressure. Ten (10) mg of the powder from the residue is placed in a covered central tube, to which 1% of citric acid methanol solution is added. After the tube settled for one hour and is shaken for 10 seconds, it is centrifuged. Supernatant is collected. 10 mg of the supernatant is added to 5 ml of the extraction supernatant solution. A sample of 1-4 μl is taken and injected into the HPLC system for analysis. A typical peak profile is obtained as shown in FIG. 7. The accurate amount is determined by using the regression equation with a correct dilution factor.

Analysis may be performed using Water HPLC system together with a U6K injector and a 490 adjustable UV/visible detector. The separation conditions are as follows: column: 3.9×2.5 cm silica gel; mobile phase of ethyl acetate, methanoic (formic) acid and ethanol at a ratio of 15:3:2; flow rate of 1.5 ml/min; detection wavelength of 346 nm; sensitivity setting of 0.01 AUFS. Window Diagram Technique is used to optimize the ratio of the mobile phase. A standard curve is prepared using the method similar to the one described above for the HPLC system. A typical regression curve in the range of 0.06-0.39 μg of berberine is: Area=2.328×10 amount (μg)−4.656×10 (r=0.9997).

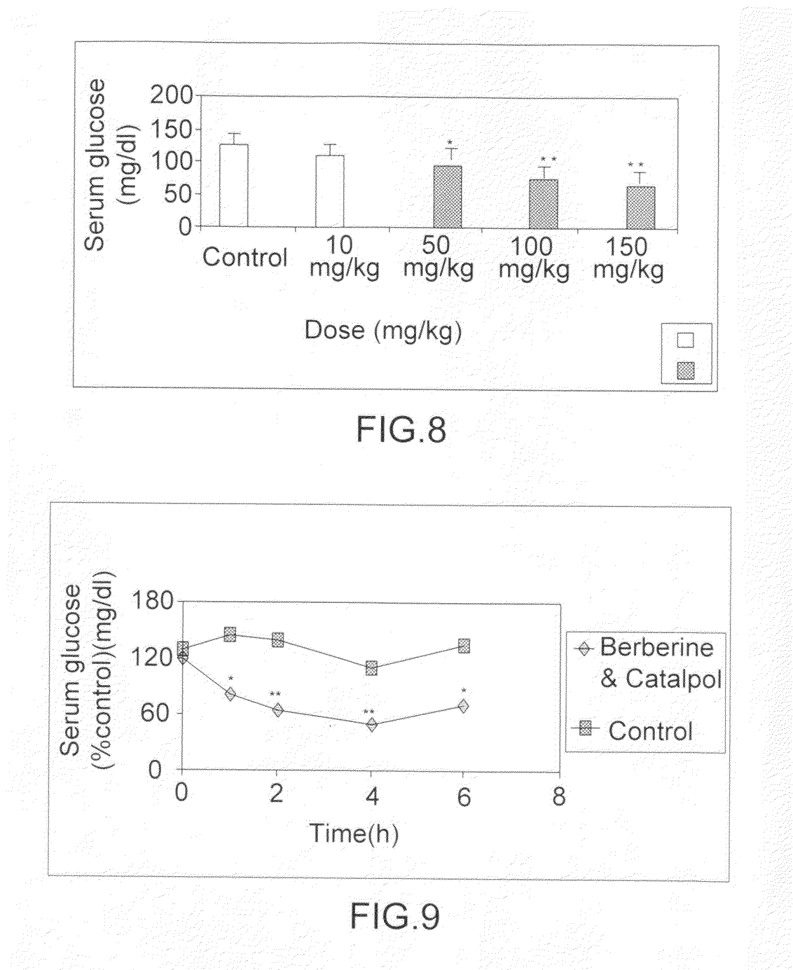

Referring to FIG. 8 of the drawings, a bar chart showing the effect of berberine and catalpol on lowering the serum glucose of normal mice is illustrated.

The effect of the berberine is the most quickly and distributed extensively, when the berberine is labeled with Hydrogen 3 and is administered to domestic rabbits by direct feeding that berberine is injected through a non-invasive tube directly through the mouth or by injection. The concentration, which is measured by radiation intensity, is the highest in the lung, and then in liver, spleen, kidney and heart. The total amount in the blood plasma is 38%±3%.

Within the six days after the injection of the labeled berberine into large mice, the amounts of the labeled berberine found in the mice's urine and feces were 73% and 10.9% of the injected concentration respectively. The labeled berberine found in the urine was primarily the original form although some of it became metabolized products. It indicates that livers and intestines were involved in the metabolism of the berberine.

In a clinical study, it was found that the plasma sugar level of the treated patients is reduced and serum insulin level is increased after the administration of berberine and catalpol. This suggests that the berberine and the catalpol are capable for reducing the plasma sugar level and for resisting the elevation of sugar hormones.

Berberine and catalpol were correlated to the restoration, regeneration and recovery of competent insulin beta (β) cells. Their capability of reducing plasma sugar level was closely related to increase blood lactic acid, which in turn would affect the insulin receptor binding affinity. The fact that berberine and catalpol have no effect on the plasma sugar level of normal mice indicates that they have the function of bi-directional adjustments.

Pharmokinetic properties of berberine are performed by conventional method involving radioactive isotope. After berberine labeled by ³H is absorbed by animals, blood sample is collected and radiation intensity is measured by using an FJ-2101 dual channels liquid scintillation counter using ppo, popop, naphthalene toluene, and ethylene glycol ethyl ether as the scintillation liquid. All samples are calibrated by using an internal standard.

To separate and quantify berberine in a blood sample or in a homogenized tissue sample, 20 μl lauryl sodium sulfate (18% vol/wt) and 0.2 ml blood sample or homogenized tissue sample are mixed in a small test tube. 300 μg quinidine is added to the test tube as an internal standard. One ml chloroform is added into the test tube, which is shaken for ten minutes. Then, the test tube is centrifuged at 400 rev/min for five minutes with a sufficient gravity for separating residues from its liquid. An absolute amount of 0.8 ml of the chloroform is collected by a pipette and is subsequently dried by blowing air. The residue from the dried chloroform is collected and dissolved in a suitable amount of dry ethanol which contains essentially no water. The sample is ready for analysis using thin layer chromatography.

A silica gel plate is cut into nine strips, each being 1.0 cm wide. Separation is conducted under running buffer of ethyl acetate, ethyl methanoate, methanol and water mixture at a ratio of 5.4:4.6:1.2:1.0. The sample size is 5 μl. When a desirable separation is achieved, the strips are scanned for reflective intensity at wavelength λₑₓ, 350 nm and λₑₘ 550 nm using Japanese Shimatsu CS-900 double wavelengths thin layer reflective straight line scanner with a Xenon lamp. The internal standard, quinidine, has λₑₓ 350 nm and λₑₙ, 450 nm. All scans are performed with 0.5 mm×10 mm slit, at scanning speed 20 mm/min, and at recording paper speed 10 mm/min. The recovery rate is about 100.3%.

After the blood serum's pH is adjusted to basic, ethyl ether is used to extract berberine. After it is dried, the residues are dissolved in a mobile phase. A fixed amount of sample is introduced into the HPLC system containing a reverse C18 column. The mobile phase is methanol, water and ethylenediamine at a ratio of 75:25:0.5. The pH is adjusted to 6.8 by using acetic acid. Concentration or amount is monitored at wavelength (λ) 345 nm.

The concentration or amount of berberine may also be determined by the following methods. Sample is extracted at 85° C. and prepared as described above and is brought into a final fixed volume. A portion of the sample is injected into the HPLC column running under the following operating conditions: ODS silica TSK gel, LS-410 5 μm column; mobile phase of lauryl sodium sulfate, 0.1M tartaric acid, methanol, and water at a ratio of 0.5:49.5:10:40; flow rate at 1.5 ml/min; and temperature at 25° C. Concentration of the sample is determined by monitoring the light intensity at the exit of the column.

Another alternative method involves a hard starch gel (s-17) separation column.

Separation is conducted by running a mobile phase consisting of water, acetonitrile, acetic acid, and triethylamine at a ratio of 80:20:0.3:0.745 at pH 8.5 at a flow rate of 0.65 ml/min under room temperature. Concentration of the amount of berberine is determined by measuring emission intensity at λ=350 nm. The amount or the concentration is determined by comparing the intensity reading with a standard curve.

Another alternative method is extraction by methanol. Solvent is removed by boiling. The residue is collected and dissolved in a small amount of hot methanol. After the methanol solution is cooled to room temperature, it is centrifuged. The methanol solution is added with 2.0 mg/ml methanol solution as an internal standard and is brought to a final fixed volume. A small amount of the sample is introduced into a Bondapa C18 separation column. The mobile phase is acetonenitrile and phosphate buffer at a ratio of 60:40 and at approximate pH of 5.2. Separation is performed at a flow rate 1.0 ml/min under room temperature. Berberine concentration is determined by monitoring the light intensity at λ=254 nm and comparing it with a standard curve obtained under the same conditions.

After the domestic rabbit is fed or injected with berberine, the concentration time curve shows a fast phase and a slow phase. Its kinetics follows open dual compartment three term model. C=Ae^{-a}+Be^{-β}−(A−B)e^{-kat}, where ka is average rate constant weighed by their coefficients: ka=(Aβ+Bα)/(A+B). The berberine concentration of the effective form increases after the administration and reaches the maximum (cₘₐₓ) at time tₘₐₓ. Thereafter, the concentration decreases as the result of elimination.

The concentration in the blood of domestic rabbits reaches the maximum about 50 minutes after berberine administration. Kinetic parameters obtained for domestic rabbits by oral administration are approximately as follows: apparent “half life” of distribution (t½ α) is 1.41±0.16 hours; apparent half life for elimination (t½ β) is 35.3±1.3 hours; apparent rate constant ka is 2.45±0.22 per hour; apparent blood berberine concentration (Vc) is 7.9±0.9 l/kg; and apparent drug distribution volume (Vd) is 20±3 1/kg. Kinetic parameters obtained from domestic rabbits by intravenous injection are approximately as follows: apparent half life of distribution (t½ α) is 1.03±0.11 hours; apparent half life for elimination (t½ β) is 35.8±2.0 hours; apparent blood berberine concentration (Vc) is 6.66±0.18 l/kg; and apparent distribution volume (Vd) is 22.1±1.71 l/kg. The data indicates that berberine is absorbed rapidly, distributed widely, and cleared up slowly.

When 300 mg of berberine is taken by human patients orally, some pharmacokinetic parameters for a single dose of berberine are found as follows: t½, the half life corresponding to apparent absorption rate constant ka, is 0.87 hour; t½, the half life corresponding to elimination rate constant Ke, is 2.94 hours; time to peak plasma concentration, tₘₐₓ is 2.37 hours; and the maximum plasma concentration Cₘₐₓ is 395 ug/l.

Method of and composition of the present invention has been used for treating diabetic living subjects. For the same reason, whenever reference is making to berberine, it also includes any of the salts as long as it is not toxic to the living subjects in a degree, that is unacceptable under given conditions. While many of the salts might have some mild side effects and or toxicity, they are within the scope of the present invention. Berberine means not just the any of the three free forms discussed above, but also its salts unless the language in the context suggests that it means only the free form. Some common salts are hydrochloric acid salt, citric acid salt, phosphate salt, and sulfuric salt.

Because berberine can exist in different free forms and salts, the dose in this disclosure and claims thereafter will refer to the quantity or amount of the free form or ammonium ion. For example, if a salt of berberine is used in a pharmaceutical composition, the dose amount would mean the amount of salts that contains these same moles of the free berberine. Approximately, it is the amount of the tertiary ammonium ion of berberine.

A preferred embodiment of the present invention is a pharmaceutical composition of the compounds for treating type II diabetes, hyperglycemia, and cholesterol elevation or abnormal cholesterol metabolisms. The composition comprises the berberine and the catalpol and a pharmaceutically acceptable carrier. The pharmaceutical composition may be administered in any effective, convenient manner to living subjects orally or parenterally, including subcutaneous, intramuscular, intradermal, and intravenous injection, or as a suppository or pessary, including topical, oral, anal, vaginal, intraperitoneral, intranasal routes among others.

The carrier must be acceptable in the sense of being compatible with other active or inactive components and all other attendant additives, and do not involve undesirable chemical reactions or cause undesirable physical transformations of any of other components. In addition, it will not be injurious to the recipients of the composition. Such carriers can be solid, powder, liquid, or gas. A solid carrier may be composite matter, which consists of more than one compounds or chemical elements. A gas carrier may contain one or more gases. A liquid carrier may be water, organic compound, oil and their combinations, or anything existing in a liquid form under normal condition. When the carrier is in form of semi solid, gel or viscous shape, it may contain solid, gas and liquid, each of which may be single compound of element or several compounds or elements.

The composition according to a preferred embodiment of the present invention, which in intended primarily for oral administration, may be presented as a draught in water or in a syrup, in capsules, cachets, boluses or tablets, as an aqueous or oleaginous solution or suspension, as suspension in a syrup, such suspension optionally including suspending agents, or as oil in water or water in oil emulsions.

The composition according to another preferred embodiment of the present invention, which is primarily in solid state, comprises the carriers, but is not limited to, of calcium phosphate, magnesium state, talc, sugars, lactose, dextrin, starch, gelatin, cellulose, methyl cellulose, sodium carboxymethyl cellulose, polyvinylpyrrolidine, low melting waxes, and ion exchange resins. The solid carrier may include one or more substances which act as flavoring agents, lubricants solubilizers, suspending agents, fillers, glidants, compression aids, binders, tablet disintegrating agents or an encapsulating material. This form of composition may be in form of tablets where the active compounds are mixed with a carrier having the necessary compression properties in suitable proportions and desired sizes and shapes.

According to another embodiment of the composition of the present invention, the composition is in powder form and the carrier is a finely divided solid, which is in admixture with the finely divided compound. Like with a solid carrier, pharmaceutical additives such as flavoring, sweetening, preserving, thickening or emulsifying agents may be included. Tablets may contain those compounds as a powder or granules optionally mixed with binders, lubricants, inert diluents or surface active or dispersing which are useful in such composition.

The composition according to another embodiment of the present invention, which primarily exists in liquid or emulsion, comprises a liquid carrier which may be used in preparing solutions, suspensions, emulsions, syrups, elixirs and pressurized foams, suspensions or emulsions. Pharmaceutically acceptable liquid carriers include water, an aqueous solution, an organic solvent such as alcohols or organic mixture, water and organic mixture, or pharmaceutically acceptable oils or fats.

A pharmaceutical composition that is sterile solutions or suspensions can be utilized by parenteral administration, such as, intramuscular, intraperitoneal or subcutaneous injection. Sterile solution can also be administered intracenously. If the composition is intended for parenteral administration, the carrier can also be an oily ester such as ethyl oleate and isopropyl myristate. The liquid carrier for pressurized final product can be halogenated hydrocarbon or other pharmaceutically acceptable propellants. The liquid carrier of the composition can contain other suitable pharmaceutical additives such as solubilizers, emulsifiers, buffers, preservatives, sweeteners, flavoring agents, suspending agents, thickening agents, colors, viscosity enhancers, stabilizers or osmotic pressure regulators if compatible.

Other embodiments of the composition may optionally comprise oleanolic acid as long as it will not incompatible with other existing compounds and additives in the composition.

Preferably, the pharmaceutical composition is in a unit dosage form, such as tablets or capsules. In such form, the composition is subdivided in unit dose containing appropriate quantities of the compound; and the unit dosage forms can be packaged compositions. For example, packet powders, vials, ampoules, pre-filled syringes or sachets containing liquids. The unit dosage from can be, for example, a capsule or tablet itself, or it can be the appropriate number (or multiplier) of any such compositions in the package form.

A dosage in the range from 1 to 300 mg/kg/dl is contemplated, with a preferred dose in a range from 0.1 to 100 mg/kg/dl. Due to the uncertainty in relating laboratory mouse study data to other mammals, condition of gravity of disease, and the compounds in the composition being selected, the dosage used for the treatment of type II diabetes must be determined by a physician or veterinarian according to standard medical or veterinary practice.

According to another preferred embodiment of the present invention, a method of using the berberine in neat form is introduced, wherein the berberine and a pharmaceutical carrier, or a composition containing the berberine and the catalpol is used for treating diabetes and peripheral complications of diabetic living subjects, including human beings. The method comprises the steps of applying any one or more the three types of agents to the living subject orally or parenterally including subcutaneous, intramuscular and intravenous injection, or as a suppository or pessary. The method may also include steps of monitoring the sugar level of the living subjects.

If the berberine is used in neat form, a special dispensing tool might be necessary to allow the living subject to get suitable dose. The second and third dose forms, such as a berberine with carrier, and the pharmaceutical composition, may further contain oleanolic acid as long as it is compatible with other existing compounds and additives.

When the neat berberine is used, the dose is in the range of 1 to 300 mg/kg/day, preferably, in the range of 5 to 100 mg/kg/day. Neat berberine may be dissolved in distilled water or sterile water, depending upon the method of administration. Berberine may be dissolved in water, teas, and other drinks of use.

When the berberine and a pharmaceutical carrier is used as a dose form in the method of treatment of diabetes and some complications of living subjects, the dose form may be in any of the forms or variations, disclosed in the above for the composition except that it does not contain catalpol. Moreover, the administration method must be comparable to the form of the composition. The dose is in the range of 1 to 300 mg/kg/day, preferably in the range of 5 to 100 mg/kg/day.

When a composition disclosed above is used, the dose is in the range of 1 to 300 mg/kg/day, preferably in the range of 5 to 100 mg/kg/day. In addition, the ratio between berberine and catalpol by weight is in the range of 1/19 to 19/1, preferably in the range of 6/1-3/2. Optionally, the composition may also contain oleanolic acid and the dose is 1 to 300 mg/kg/day as long as it is compatible with other existing compounds and additives. Likewise, the composition must be compatible with the administration method.

The method of treating diabetes and some complications are further described in the following examples.

The berberine and the catalpol are water soluble and can also be easily dissolved in organic solvents such as alcohols and acetones. They are stable in nature. In the following examples, the pharmaceutical solution is prepared by dissolving berberine and catalpol in a buffer in a ratio of three to one (3:1) by weight. The pH is not adjusted. The total concentration is 20 mg/ml unless otherwise specified and provided.

In the following examples, the procedures are common when applicable. Plasma sugar is measured by glucose enzyme peroxide method using “Ames Blood Biochemical Apparatus EY-83”. In all examples, the compositions of the present invention containing berberine, catalpol and/or oleanolic acid are injected into the oral track of mice by a syringe equipped with a non-invasive tube.

Normal mice are induced to be diabetic. Normal Km male mice, weighing 20 to 25 grams, each received tetraoxide alloxan solution by occyx injection at the dose of 100 to 105 mg/kg. Seven hours after the injection and two hours after fasting, blood is drawn to determine plasma glucose levels. The mice are selected according to specific criteria, such as the plasma sugar levels, used in each of the studies.

It should also be noted that blood gluscose and blood sugar are widely used in an interchangeable way.

### Example 1

### Effects of the Berberine and the Catalpol on Plasma Sugar Level of Tetraoxide Alloxan Diabetic Mice

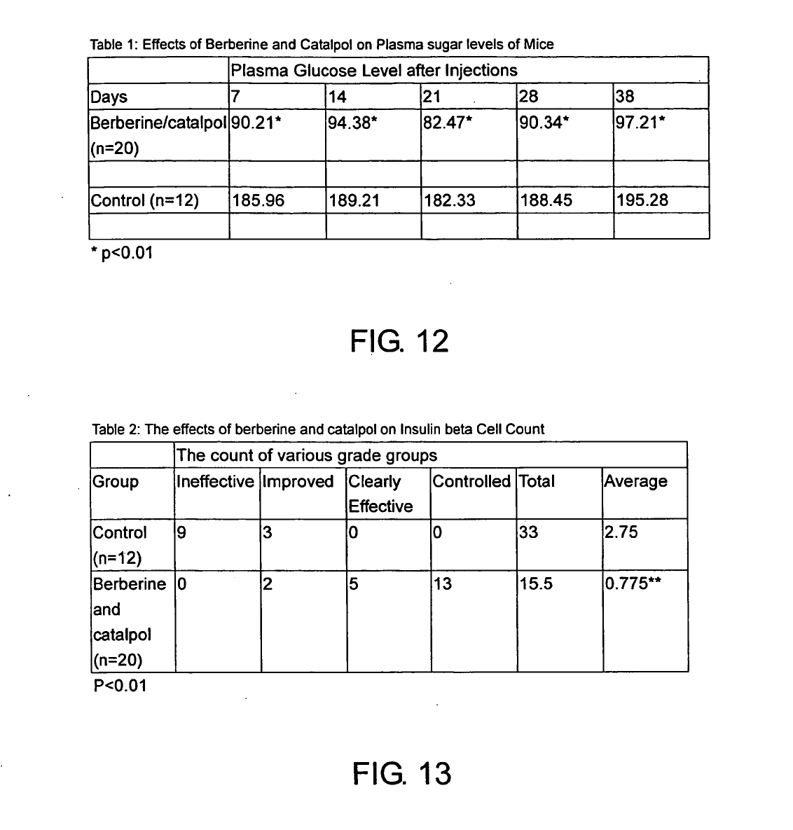

FIG. 12 is a table showing effects of the berberine and catalpol on the plasma sugar level of mice. Fifty (50) large mice of 120 to 140 grams were used The mice, with same number of both sexes, were fed in separate cages according to their sexes. The average plasma sugar level of normal mice was 120 mg/dl. Fresh 4% tetraoxide alloxan physiologic salt solution was used to make the mice diabetic. Each mouse received the tetraoxide alloxan solution at a daily dose of 12 mg/100 g. The drugs were administered through intra-abdominal injection after the mice had been fasted. Plasma sugar levels were determined on the 5^{th} day after the injection.

The plasma sugar levels of the tetraoxide alloxan diabetic mice were from 220 to 1120 mg/dl. Thirty-two of the mice were selected by eliminating those that had plasma sugar levels higher than 500 mg/dl or lower than 300 mg/dl. The selected mice were then randomly divided into two groups: 20, plasma sugar level: 421±92.5 mg/dl, for the treatment, and 12, plasma sugar level: 400±65 mg/dl for a control. The mice of the control received only 0.1 ml physiosaline solution daily through intramuscular injection for 30 days. The berberine and catalpol solution was injected into the mice of the treatment group intramuscularly at the dose of 0.1 ml, equivalent to 2 mg of the compounds, daily for 30 days.

Urine and blood were taken for sugar analysis once a week for four weeks. Ten days after the four weeks experiment, blood was drawn from the mice for plasma sugar analysis immediately after the mice were killed. The results showed that the average plasma sugar level of the treatment group was significantly lower than the control group. The difference was statistically significant with p<0.01. The effect remained ten days after the administration of the berberine and the catalpol.

### Example 2

### The Effects of Berberine and Catalpol on Restoring Insulin Beta Cells

The whole pancreases of the mice were collected after the mice were killed at the end of the experiment in example 1. Bouin picric acid was used for the fixing of pancreases before slicing was performed using the paraffin method. One slice was made for each of the pancreases for the purpose of determining insulin beta cell numbers. The slices were then dyed using aldehydes before they were placed under a microscope for examination. Insulin beta cells were counted from left to right. Exponential Notation Grade was assigned to each pancreas slice on the basis of the number of beta cells observed, according to the following scheme:

| | | |
|---|---|---|
| 3 | Points | Ineffective if insulin beta cells are |
| | | not visible or only a few are visible |
| 2 | Points | Improved if ½ of normal insulin |
| | | beta cells are visible |
| 1 | Points | Clearly effective if ⅔ or normal |
| | | insulin beta cells are visible |
| 0.5 | Points | Controlled if it is near normal |

FIG. 13 is a table shows the effects of the berberine and the catalpol on Insulin beta cell count. The Exponential Notation Grade for the berberine and catalpol treatment is significantly lower than that of the control (p<0.01). The study indicates that the berberine and the catalpol are effective in restoring insulin beta cells of the mice (p<0.01).

### Example 3

### The Effects of Berberine and Catalpol on the Plasma Sugar Level of Normal Mice

Sixty (60) normal mice were divided equally into five groups. One group was used as control and one as a treatment. The mice take a composition of the berberine and the catalpol by oral administration after they had been fasted for two hours. The berberine and the catalpol doses used were 10, 50, 100 and 150 mg/kg, respectively. Blood was drawn from the back of the eye sockets of the mice at the designated times.

Referring to FIG. 8 of the drawings, the composition of the berberine and the catalpol at a dose of 50 mg/kg was able to lower the plasma sugar level of the normal mice. The related anti-hyperglycemic effect was also clearly shown.

### Example 4

### Effect of Berberine and Catalpol Administration Frequency on the Plasma Sugar Level of Normal Mice

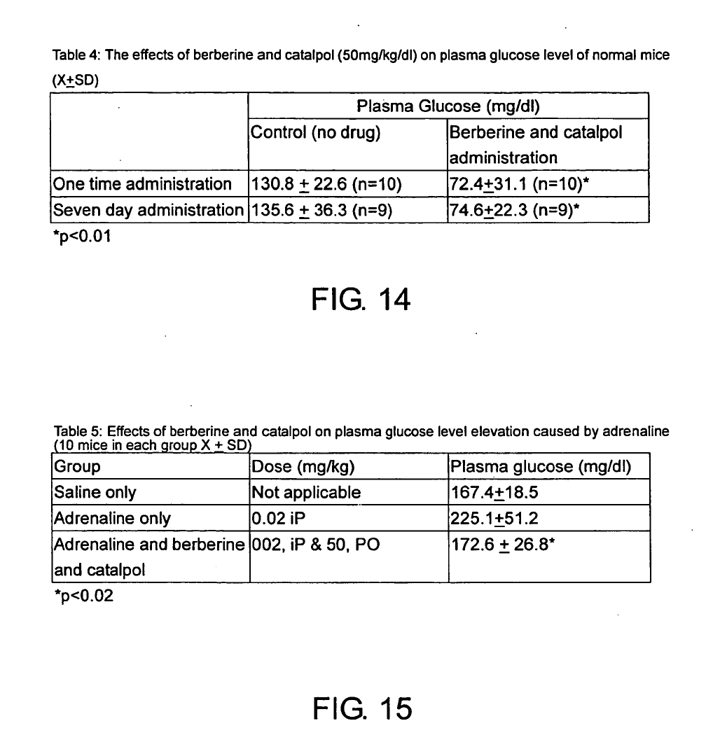

FIG. 14 is a table showing the effects of the berberine and the catalpol (50 mg/kg/dl) on plasma glucose level of normal mice (X±SD). The study was done as in the example 3 with normal mice except that treatment was one time administration or seven day successive administration of the berberine and catalpol solution. Two corresponding control groups (n=9 and n=10, respectively) received no drugs. Both the one time administration group (n=10) and the seven day administration group had lower plasma sugar level. The difference in plasma sugar level between the treatment group and the control was statistically significant, regardless of the administrative frequencies (p<0.01).

### Example 5

### Effective Duration of Berberine And Catalpol on Lowering Plasma Sugar Level of Normal Mice

Nine groups of normal mice, ten in each group, were used in this study. Five groups of the mice received berberine and catalpol by oral administration at a dose of 50 mg/kg after they had been fasted. The other four groups were used as control. Blood was drawn from the back of eye-sockets of the mice for plasma sugar analysis at 1, 2, 4 and 6 hours after the oral administration. The plasma sugar levels immediately before the drug administration were treated as the zero hour values.

Referring to FIG. 9 of the drawings, the average plasma sugar levels of the mice over time for both the five treatment groups and the four control groups are shown. The average percentages of the plasma sugar levels were computed for the treatment groups, relative to the four controls. The average plasma sugar level of the treatment groups was lower significantly one hour after berberine and catalpol were administered. The effect of berberine and catalpol on lowering plasma sugar level of the normal mice was greatest between 2 and 4 hours after the drug administration.

### Example 6

### The Effect of Berberine and Catalpol on Preventing Plasma Glucose Elevation of Normal Mice Caused by an External Source

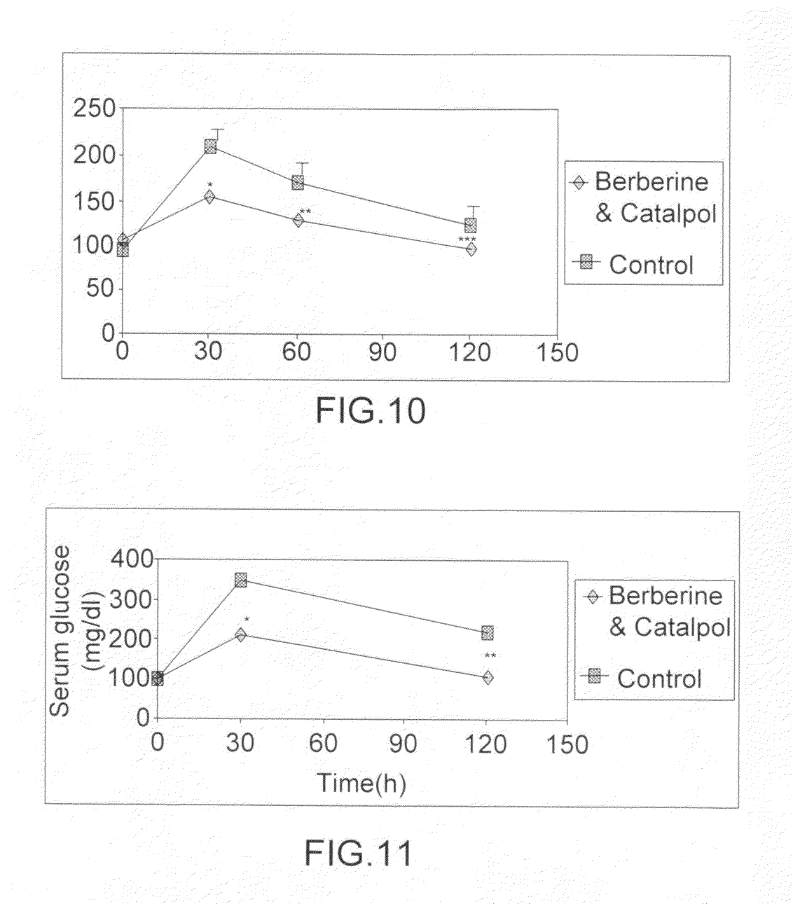

Seven groups of normal mice, each having 10 mice, were used in this study. After the mice had been fasted for four hours, their blood was taken for plasma sugar analysis for all groups. The values of the plasma sugar levels were regarded as the zero hour plasma sugar levels. Three groups were used as treatments, to which the berberine and the catalpol were administered orally at a dose of 50 mg/kg. The other three groups were used as control. After drug administration with the three treatment groups, all mice were immediately injected in abdomens with a glucose solution at a dose of 2.0 gm/kg. Blood was drawn from the back of the eye-sockets of the mice for plasma sugar analysis at 30, 60, and 120 minutes after the glucose injection. Referring to FIG. 10 of the drawings, the berberine and catalpol were capable of resisting the elevation of plasma sugar from the external source.

### Example 7

### Effect of Berberine and Catalpol on Adrenaline Plasma Sugar Level of Normal Mice

Thirty normal mice were divided into three groups with equal number. After two hours of fasting, one group of the mice received berberine and catalpol by oral administration at a dose of 50 mg/kg, and one group was a control receiving no administration. After one hour, hydrochloric adrenaline solution at a concentration of 20 mg/kg was injected into the abdomens of the mice for both the treatment groups and the control group. The third group was given abdominal injections of physiosaline solution. Blood was drawn from the back of the eye sockets of the mice for plasma sugar analysis 30 minutes after the injection of adrenaline solution.

Referring to FIG. 15 of the drawings which is a table showing effects of berberine and catalpol on plasma glucose level elevation caused by adrenaline (10 mice in each group X±SD), the injection of the adrenaline solution caused the plasma sugar level to increase from 167 to 225 mg/dl. However, the berberine and the catalpol were able to resist adrenal gland's plasma sugar elevation and to maintain the plasma sugar level close to the level of the normal mice.

### Example 8

### Effect of Berberine and Catalpol on Plasma Sugar of Tetraoxide Alloxan Diabetic Mice

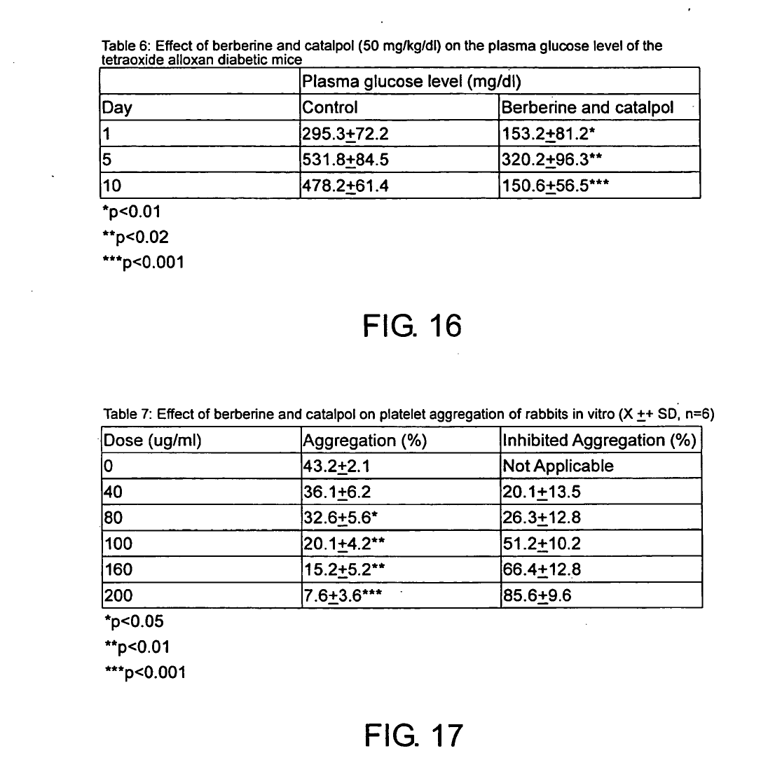

Tetraoxide alloxan was injected into the occyx veins of normal mice to make them diabetic. The treatment group, each having 10 mice, received the berberine and the catalpol by oral administration at a concentration of 50 mg/kg/dl while a control group, each having 10 mice, received no drug. Referring to FIG. 16 of the drawings which is a table showing effect of berberine and catalpol (50 mg/kg/dl) on the plasma glucose level of the tetroxide alloxan diabetic mice (10 mice in each group, each data point represent X±SD), the plasma glucose level of the mice on the 1^{st}, 5^{th}, or 10^{th} day after the drug administration are shown. The berberine and the catalpol were able to reduce the plasma sugar levels of the tetraoxide alloxan diabetic mice significantly with p<0.01 or p<0.001.

### Example 9

### Effect of Berberine and Catalpol on the Plasma Sugar Level of Spontaneously Diabetic kk Mice

Sixteen spontaneously diabetic kk mice, weighing 25 to 30 grams, each within a mixture of both sexes, were used. The mice, each fed with dairy products, were divided into two groups evenly. One group was a control that received no drug. A treatment group received berberine and catalpol by oral administration at a dose of 50 mg/kg/dl for twenty days, successively. Blood was drawn from the back of the eye-sockets of the mice for plasma sugar analysis. The plasma sugar level of the control group was 90.5±20.0 mg/dl while the plasma sugar level of the treatment group was 70.5±12.5 mg/dl. The results show that the plasma sugar level of the mice was lowered by 35% on average after the mice had received berberine and catalpol for 15 days. The difference was statistically significant with p<0.01.

### Example 10

### Effect of Berberine and Catalpol on the Glucose Tolerance of Spontaneously Diabetic kk Mice

Thirty two spontaneously diabetic kk mice were divided into four groups, each having eight mice. Two groups, after four hours food deprivation, received the berberine and the catalpol oral administration at a dose of 50 mg/kg/dl for 20 days, successively. The other two groups were untreated control. In the last drug administration, the mice were fasted for two hours. Blood was drawn from the back of the eye-sockets of the mice for plasma sugar analysis for one treatment group and one control group. The plasma sugar levels were treated as the zero hour values respectively, for the treatment and control group. Another treatment group and another control group, glucose at a dose of 2 gm/kg was injected into the abdomens of the mice immediately after the drug administration. Blood was drawn from the back of the eye sockets of the mice for plasma sugar analysis at 30 and 120 minutes after the injection of glucose. Referring to FIG. 11 of the drawings, the plasma sugar levels of the treatment group and the control group were substantially similar at zero hour. After glucose injection, plasma sugar elevation in the treatment group was smaller than that observed for the control group. This suggests that berberine and catalpol were able to improve the glucose tolerance of the spontaneously diabetic kk mice.

### Example 11

### Effect of Berberine and Catalpol on the Blood Cholesterol Level of Normal Mice

Twenty mice were divided into two groups, each group having 10 mice. All mice received high cholesterol emulsion in the amount of 0.5 ml by orally in the afternoon to raise their blood cholesterol levels. One group of the mice received berberine and catalpol by oral administration at the dose of 50 mg/kg/day in the morning. The other group was a control. The experiment was carried out for seven days successively and blood cholesterol levels were analyzed after the mice were fasted for four hours. The blood cholesterol level of the control group was 498.8±82.4 mg/dl, and the blood cholesterol level of the treatment group was 201.3±32.2 mg/dl. The results show that after seven days of the berberine and the catalpol administration, the blood cholesterol level of mice, which was elevated by feeding high cholesterol emulsion, was significantly lower with p<0.001.

### Example 12

### Effect of Berberine and Catalpol on Blood Platelet Aggregation of Domestic Rabbits

Six domestic rabbits, each having a weight in a range of 2 to 2.5 kg, were used in Example 12. The blood platelet aggregation of each rabbit was observed. ADP's doses of 2 to 10 umol were chosen so that ADP was able to cause platelet aggregation by 40% to 50%. The doses of berberine and catalpol were 40, 80, 120, 160, and 200 ug/ml (final concentration) respectively. Each of the doses was measured and delivered using a parallel twin-tube which was able to suck in a given amount of solution by capillary action. The average value f polymerization rate was determined using double channel platelet polymerization apparatus. Referring to FIG. 17 of the drawings which is a table showing effect of berberine and catalpol on platelet aggregration of rabbits in vitro (X±SD, n=6), the results show that the berberine and the catalpol are able to inhibit blood platelet aggregation induced by ADP.

### Example 13

### Clinical Study of Curative Efficacy of Berberine and Catalpol on type II Diabetes and Some Complications

In example 13, one hundred cases of insulin-independent diabetes mellitus were selected according to the diagnostic criteria and classification scheme of World Health Organization. Among the cases, 62 were male and 38 were female. Their ages ranged from 36 to 72 with an average of 55. The courses of disease of the patients were from 3 months to 6 years. None of the patients received insulin as a supplementary treatment, and none of them suffered from ketoacidosis. Among all the cases, 30 were severe diabetic having a fasting blood glucose level higher than 250 mg/dl, 60 were medium diabetic having a fasting blood glucose level between 150 and 250 mg/dl, and 10 were light diabetic having a fasting blood glucose level less than 150 mg/dl. Among all the cases, 15 had synthesized hypertension, 5 had coronary heart disease, and 80 had inexplicit complications. The patients were randomly divided into two groups, a treatment group having 60 cases and a control group having 40 cases. An additional 40 cases with normal plasma sugar level were used as a normal group in order to understand whether berberine and catalpol have a bi-directional adjustment and the side effect of causing hypoglycemia. Before any treatment was applied, plasma sugar level and cholesterol level of the treatment and the control groups were not significantly different with p>0.05.

The berberine and the catalpol were taken orally by the 60 cases in the treatment group, each at a dose of 300 mg for three times a day. Every single dose was appropriately adjusted according to the plasma sugar level before meal was taken according to the following criteria. If fasting plasma sugar level was less than 8.33 mmol/l (150 mg/dl), and was between 8.33 and 13.9 mmol/l (250 mmol/l), the dose was 200 mg and 300 mg respectively. The cases in the control group only used dietary management wherein 5 mg glucophage or 15 mg actos was used daily or twice a day respectively. No other medication was used to lower cholesterol and affect blood coagulation for the control group. The trial in the example 13 lasted two months.

Observations was made to seven indexes, including (1) fasting plasma sugar level before and after treatment (FPG), (2) Apolipoprotein Al (APOAI), (3) Apolipoprotein B (APOB), (4) total cholesterol (TC), (5) Triglyceride (TG), (6) High density lipoprotein (HDL), and (7) Low density lipoprotein (LDL).

In addition, hematological properties of blood were also determined. A whole blood viscosity (mPa-S) of each case was measured by using Taber Board Model Viscosity Measurement Mode. The whole blood viscosity was between 5.19 and 6.70 mPa-S for male cases and was between 4.13 and 5.50 mPa-S for the female cases. Blood plasma viscosity was between 1.56 and 1.75 mPa-S, fibrinogen (Fg) determined by using double contraction urine method was between 2.0 and 4.0 g/l, and red cell content was between 43% and 47% for the male cases and 36% and 40% for the female cases.

The average of each index and its corresponding standard deviation (SD) was computed for each of the treatment, the control and the normal groups. Comparisons of the average values were made using student test to determine if there was a significant difference in the average value of each index before and after treatment.

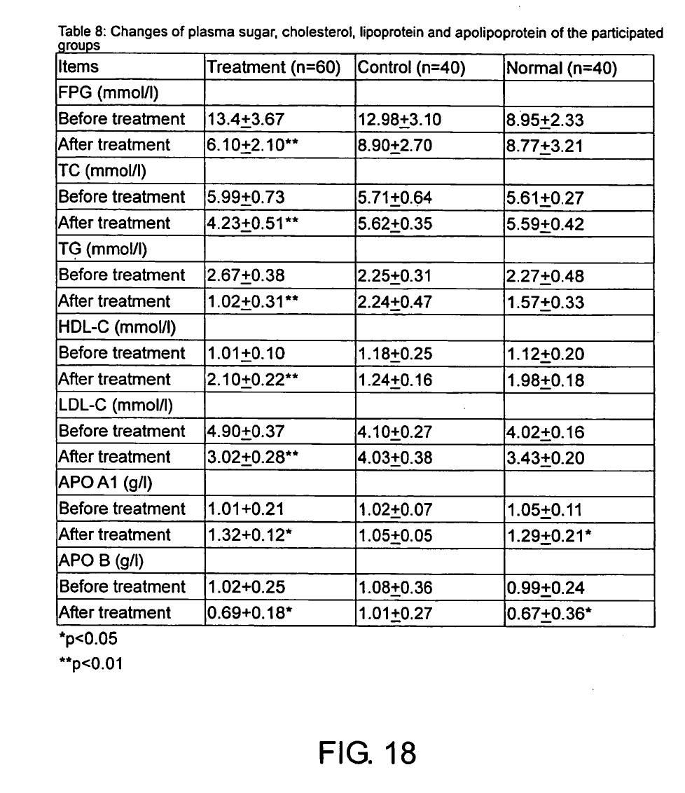

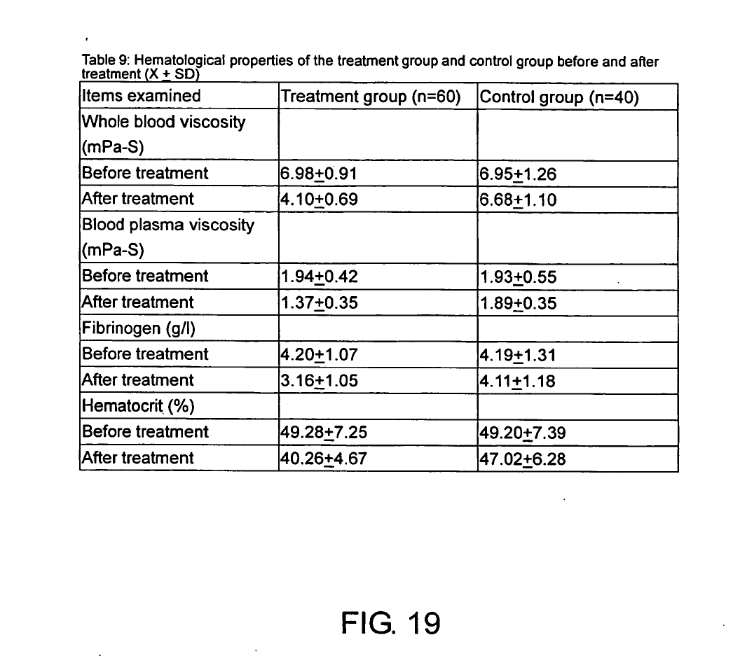

The results are shown in FIG. 18 and FIG. 19 of the drawings, which are tables showing changes of plasma sugar, cholesterol, lipoprotein and apolipoprotein of the participated groups and showing hematological properties of the treatment group and control group before and after treatment (X±SD) respectively, the berberine and the catalpol were able to lower TC, TG, LDL and APOB significantly but to raise HDL and APOA1 significantly. The berberine and the catalpol were able to significantly reduce the whole blood viscosity, the blood plasma viscosity, the fibrinogen content, and the red cell content with p<0.01 or p<0.05. The difference of each of the index between the control group and the normal group was statistically insignificant. The results suggest that the berberine and the catalpol would not cause hypoglycemia of the normal people and have the function of dual direction adjustments. The effectiveness rate is close to 96% without showing observable side effects.

The Example 13 indicates that high density lipoprotein transported excessive cholesterol of external peripheral tissues to the liver and most of it is transformed into fatty acid showing anti-AS effect. The apolipoprotein A1 exists mainly within HDL. Anti-AS is a factor which takes part in the reverse transport of TC from external peripheral tissues to the liver. APOB exists chiefly with LDL and it guides TC to external tissues and increased the sediment of LDL on the internal walls of arteries which is the leading cause of AS.

In additional to the above three active ingredients, the berberine, the catalpol and the oleanolic acid, the present invention may further comprises a predetermined supplementary composition. The supplementary composition is gerenally having an effect of lowering the blood sugar and the cholesterol level, and having an effect of anti-obesity and anti-inflammation. These effect is beneficiary to the diabetics and may assist to enhance the body conditions. The supplementary composition includes transhinone I, transhinone IIa, salviol, dioscin, diosgenin, phosphoenolpyruvate carboxylase, ophiopogonin A, ophiopogonin B, ophiopogonin C, ophiopogonin D, chrysophanol, emodin, taurine, alyinic, laminarin, anemarans B, and panaxans. It is known that the above compositions may be obtained from different herbs. For example, transhinone may be obtained from Danshen, Emodin may be obtained from Rhei Rhizome, Anemarans may be obtained from Anemarrhena aspodeloidea bunge. The source of the supplementary composition are not mentioned as it is well known to the skilled person in the field of the present invention.

Accordingly, the present invention provides a method of treating of a living subject with non-insulin dependent diabetes mellitus, comprising a step of administrating to the living object a composition comprising a berberine as a first active ingredient and a catalpol as a second active ingredient, wherein the composition may further comprise an oleanolic acid as a third active ingredient.

The present invention also provides a composition of treating non-insulin dependent diabetes and related complications, comprising a berberine as a first active ingredient and a catalpol as a second active ingredient, wherein the composition may further comprise an oleanolic acid as a third active ingredient.

In one embodiment, the composition is prepared into a predetermined form for administration that contains 1 to 300 mg/kg/dl of the berberine, and preferably that contains 5 to 150 mg/kg/dl of the berberine. The form for administration includes draught in water, syrup, caches, tablet and solution.

Alternately, the composition is prepared into a predetermined form for administration that contains 1 to 300 mg/kg/dl of the first and second active ingredients, or the first, second and third active ingredients.

The present invention further provides a method of treating of a living object with a disease selected from the group consisting of insulin independent diabetes, cholesterol elevation, and hyperglycemia, wherein the method comprises a step of: administrating to the living object a pharmaceutical composition containing an active compound selected from the group consisting of a barbering and salts of barbering in a therapeutically effective dose in a pharmaceutically acceptable carrier to the living object.

In particular, the dose of berberine is in a range of 1-300 mg·kg/dl, and preferably in a range of 5-100 mg/kg/dl.

The berberine is preferably obtained from one or more natural herbs selected from the group consisting of Berberis, Chelidonium, Stephniz, Coptis, Phellodendron, and Ziziphus. The catalpol is preferably obtained from one or more natural herbs selected from the group consisting of Rehmannia, Verbascum, Panulownia, Glubularia, and Adonis. The oleanolic acid is preferably obtained from one or more natural herbs selected from the group consisting of Olea, Swertia, Astrantia, Lonicera, and Beta.

In particular, the berberine is extracted by the steps of:

(a) providing a sample having the berberine;

(b) soaking the sample with ethanol to form a mixture and preparing a concentrated mixture solution from the mixture;

(c) filtering the concentrated mixture solution after an equilibrium of the concentrated mixture solution is established;

(d) obtaining a filtrate solution from step (c);

(e) extracting participates from the filtrate solution by rinsing the filtrate solution with an acid; and

(f) obtaining the berberine from the participates.

The examples described above are not intended as the limitations to the scope of the present invention. The details are provided as the guidance in using the method in various laboratory and clinical conditions. It will be understood that various modifications in the method and the composition such as relative ratio and doses may be made in the exemplary method and compositions without departing from the scope of the invention.

Additional effect of the method of treatment and the composition is further demonstrated by the following clinical observation in which 24 cases are observed in compliance with the WHO Diagnosis Standard.

Clinical Observation of Diabetic Limb-End Gangrene Treated 24 Cases with “Glucosynergy”

Diabetic limb-ends gangrene is one of the diabetic vascular diseases. It takes place when the limb-ends contracted necrosis caused by shortage of blood. Thus, it is also called “the diabetic foot disease”. Its main clinical symptom manifests in early stage of numbness, pain and coolness. Intermittent lameness is obvious. It is, then, followed by skin turning black, tissues ulcerating, infection, and gangrene. The more severe one would lead to complication of osteomyelitis. When this disease occurs, it develops very fast and its symptom worsens. Patient's condition can no longer be controlled by treatment of simple western medicines and the possibility of amputation is very high.

The subject study is about employing “Glucosynergy” to effectively control the patient's blood sugar level, to treat the diabetic limb-end gangrene. It is specially used in the early stage of treatment. It significantly shortens the course of disease enhances the curative rate, and minimizes the occurrence of invalid.

I. Clinical Information

1. General Information

According to the Diagnosis Standard of WHO, 24 cases of limb-end gangrene of diabetic complication are randomly selected and divided into Treatment Group and Western Medicine Group.

a) 18 Cases of Treatment Group, 12 cases are male and 6 cases are female. Age range is 46˜70 and average age is 55. Course of diabetic disease is 3˜15 years and averaging 6.5 years. Course of limb-end gangrene disease is 1˜11 months and averaging 6 months.

b) 6 cases of Contrast Group, 4 cases are male and 2 cases are female. Age range is 44˜70 and average age is 56. Course of diabetic disease is 3˜13 years and averaging 6 years. Course of limb-end gangrene disease is 1˜10 months and averaging 4.5 month.

2. Classification of Disease Condition

All Cases are equally divided as patients of complication of Diabetes Type II limb-end gangrene, among which, 2 cases are of “dry” gangrenes, 22 cases of “wet” gangrene and 1 case of severe abscess. 5 cases have the area of ulceration of 1-3 cm; 2 cases 3-5 cm; 1 case more than 5 cm.

Similar condition and extent of gangrene of both groups are basically the same. They are comparable.

II. Methods of Treatment and Observation

1. Treatment Group Uses: “Glucosynergy” (3×600 mg/3 Times Daily)

Contrast Group employs blood vessel dilation and anti-infective medication in treatment.

Contrast Group Use: “Glucophage” (5 mg/Daily)

2. Index of Observation

Blood sugar, uric sugar and diagram of bodily blood flow of all patients are examined. Some of the cases employ test of hematological later development and X-ray on toe bones. Focal points of clinical symptom are on observing the limbs' numbness, coolness, pain, and arterial pulses of instep, change of toe color and gangrene area, before and after treatment.

III. Analysis of Curative Effect

1. Criterion of Curative Effect

a) Completely recovered: abscess healed, blood sugar normalizes, uric sugar is negative; blood flow capacity of limbs is tested normal.

b) Improved: area of abscess reduces by >50%; the ultra-normal value part of blood sugar reduces by >50%.

Uric sugar level reduces by >50%; blood flow capacity of limbs clearly increases more than pre-treatment. Clinical symptom improves significantly.

c) Infective: abscess area, blood sugar level, uric sugar, determination of blood flow capacity of limbs and bodily symptom remain unchanged.

2. Result of Treatment

a) After treatment, 14 out of 18 cases of Treatment Group recover and 4 improve. Average time of recovery is 4˜6 weeks.

b) Out of 6 cases of Contrast Group, 1 recovers, 2 improve and 3 are ineffective. According to statistical process, curative effect of Treatment Group is obviously better than Contrast Group's (p<0.01)

3. Analysis of Curative Effect on Blood Sugar and Uric Sugar

Before treatment, empty stomach blood sugar and uric sugar are basically the same.

TABLE II
Transformation of 24 Hours Fixed Quantity of Uric
Sugar Before and After Treatment (x ± s) g/24 h

| | | Before | After | Value of |
|---|---|---|---|---|
| Group | n | Treatment | Treatment | Difference |
| Observation | 18 | 15.36 ± 2.96 | 7.32 ± 1.51 | 8.04 ± 1.21 |
| Group | | | | |
| Contrast | 6 | 15.23 ± 3.02 | 8.37 ± 1.89 | 56.86 ± 1.27 |
| Group | | | | |

FIG. 1 and Table II show, after treatment, FBG and 24 hours fixed quantity of uric sugar of Observation Group drop significantly. Comparing to itself of pre-treatment, after it is examined with t, it shows the difference is very obvious (p<0.01). Comparing to Contrast Group, the difference has distinct meaning (p<0.05). It indicates “Glucosynergy” is clearly able to reduce the blood sugar level and uric sugar. Its effect is better than “Glucophage”.

4. Comparison of Electrocardiogram's Hypertrophic Cardiomyopathy (see Table III)

70 cases of Observation Group; 49 are outstandingly effective, 20 effective and 1 not effective. Total effective rate is 98.6%.

50 cases of Contrast Group; 3 are outstandingly effective, 12 effective and 35 not effective. Total effective rate is 30%.

TABLE III
Transformation of Cholesterol Before and After Treatment (x ± s) mmol/L

| Group | | TC | TG | HDL | LDL |
|---|---|---|---|---|---|
| Observation Gr. | B/Treatment | 5.86 ± 1.20 | 3.58 ± 0.68 | 1.06 ± 0.14 | 5.29 ± 1.16 |
| (n = 18) | A/Treatment | 4.37 ± 0.69 | 1.08 ± 1.02 | 1.42 ± 0.28 | 3.10 ± 0.70 |
| Contrast Gr. | B/Treatment | 5.89 ± 1.17 | 3.54 ± 0.67 | 1.07 ± 0.20 | 5.27 ± 1.24 |
| (n = 6) | A/Treatment | 5.40 ± 0.76 | 3.39 ± 0.76 | 1.12 ± 0.24 | 4.51 ± 0.81 |

*B = Before,
*A = After

Table III shows after treatment, TC, TG, HDL and LDL of both groups experience changes of different degree. Comparing Observation Group to itself before treatment, elevation of HDL and dropping of TC, TG, LDL is obvious (p<0.01).

Contrast Group has certain function of lowering TG and LDL (p<0.05), but effect of dropping TG and elevating HDL is not obvious (p>0.05). Comparison of lowering TG and elevating HDL between the 2 groups, the difference is very outstanding (p<0.01). It shows “Glucosynergy” have the excellent effect of adjusting the disorder of cholesterol metabolism, whereas the effect of “Glucophage” is relatively weaker.

5. Change of Serum SOD Vitality (see Table IV)

TABLE IV
Change of Serum SOD Vitality (x ± s) nu/L

| | | Before | After | Value of |
|---|---|---|---|---|
| Group | n | Treatment | Treatment | Difference |
| Observation | 18 | 91.07 ± 16.18 | 118.78 ± 18.28 | −27.71 ± 7.21 |
| Group | | | | |
| Contrast | 6 | 89.96 ± 18.21 | 94.21 ± 17.03 | −4.25 ± 3.33 |
| Group | | | | |

Table IV shows Comparing Observation Group to itself of pre-treatment and post-treatment, the difference is very obvious (p<0.01), but comparing pre-treatment and post-treatment of Contrast Group, the effect is not obvious (p<0.05). Comparison between both group shows significant difference (p<0.01). It indicates “Glucosynergy” have very outstanding function of enhancing the vitality of serum SOD.

6. Change of Index of Hematology Later Development (see Table V)

TABLE V
Change of Index of Later Development of Serum Before and After Treatment (x ± s)

| | | Total Blood Viscosity | | | Viscosity Red Cells | |
|---|---|---|---|---|---|---|
| | | (mpa · s) | | | Accumulation | Fibrinogen |
| Group | | High Excise | Low Excise | Plasma | (%) | (q/L) |
| Observation Gr. | B/T | 6.22 ± 0.63 | 15.18 ± 2.02 | 2.78 ± 0.42 | 49.48 ± 3.21 | 4.12 ± 0.51 |
| n = 18 | A/T | 5.30 ± 0.21 | 13.02 ± 1.07 | 2.02 ± 0.30 | 45.01 ± 3.29 | 3.20 ± 0.62 |
| Contrast | B/T | 6.23 ± 0.57 | 15.21 ± 2.17 | 2.72 ± 0.66 | 49.58 ± 4.34 | 3.90 ± 0.81 |
| n = 6 | A/T | 6.33 ± 0.34 | 15.19 ± 2.10 | 2.45 ± 0.37 | 49.50 ± 4.86 | 3.45 ± 0.44 |

*B/T = Before Treatment
*A/T = After Treatment

Table V shows every hematological index of later development of both groups' patients is clearly higher than normal persons. It indicates blood of patient of diabetic coronary heart disease appears to be highly coagulated and in the state of hematoma.

After treatment, all index of Observation Group fall significantly. Comparing to itself of pre-treatment, the difference is very outstanding (p<0.01). Post-treatment of Contrast Group only presents difference of blood plasma viscosity and fibrinogen, besides that, all other index has no obvious change (p<0.05).

It indicates “Glucosynergy” have the capability to improve the index of hematological later development, to better the state of blood viscosity and to perfect the microcirculation.

7. Effect on the Experiment of Serum Insulin Release (see FIG. VI).

Table VI. Change of Value of Serum Insulin Release Before and After Treatemnt (x±s), (mu/L)

Table VI shows, after the patient has been treated with ‘Glucosynergy’, comparing the release curve of the patient's insulin to pre-treatment's, the difference is outstanding (p<0.01).

It shows ‘Glucosynergy’ is able to significantly improve the competence of insulin J3 cells.

V. Discussion

Clinical research reveals “Glucosynergy” is able to reduce the FBG of patient and the level of 24 hours fixed quantity of uric sugar. Its function lies in restoring the mechanism, in strengthening the sensitiveness of the insulin receptive-body and in improving the resistance condition of insulin.

“Glucosynergy” is able to lower the level of TC, TG, LCL, to raise the level of HDL and to enhance the vitality of SOD. As a result, the disorder of cholesterol metabolism is redressed, sediment of adipose of the artery walls reduced or eliminated, and the coronary heart disease is prevented.

This subject study shows “Glucosynergy” clearly improve the hypertrophic cardiomyopathy and abnormal electrocardiogram symptoms, such as dull-pain, tightness of chest and shortness of breath of the patient. Thus, it indicates “Glucosynergy” have the capability of eliminating fatty sediment in the vascular wall, of dilating the coronary artery and of improving the blood supply to the myocardium. They have excellent curative effect on treating diabetic cardiovascular disease.

It is worth mentioning that the present invention has a significant effect in restoring insulin beta cells which is particular important to the living object being treated when the use of insulin can no longer provide an effective treatment.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method of restoring insulin beta cells in a living object to lower a plasma sugar level, which comprises the steps of: administering a composition to the object, wherein said composition has active ingredients consisting of a first active ingredient berberine and a second active ingredient catalpol.

2. The method, as recited in claim 1, wherein said composition is manufactured into a unit dosage form having a dosage of active ingredients within a range between 1 to 300 mg/kg/dl for a single intake.

3. The method, as recited in claim 1, wherein said composition is manufactured into a unit dosage form having a dosage of active ingredients within a range between 5 to 150 mg/kg/dl for a single intake.

4. The method, as recited in claim 2, wherein said first active ingredient berberine and said second active ingredient catalpol has a relative ratio within a range from 1:20 to 20:1 by weight.

5. The method, as recited in claim 3, wherein said first active ingredient berberine and said second active ingredient catalpol has a relative ratio within a range from 1:20 to 20:1 by weight.

6. The method, as recited in claim 4, wherein said unit dosage is in a form of packet powders, solution in vails, ampoules or pre-filled syringes, capsule, tablet, draught in water, syrup or cachets.

7. The method, as recited in claim 5, wherein said unit dosage is in a form of packet powders, solution in vials, ampoules or pre-filled syringes, capsule, tablet, draught in water, syrup or cachets.

8. The method, as recited in claim 3, wherein said first active ingredient berberine is extracted from one or more natural herbs selected from the group consisting of Berberis, Chelidonium, Stephniz, Coptis, Phellodendron, and Ziziphus, wherein said second active ingredient catalpol is extracted from one or more natural herbs selected from the group consisting of Rehmannia, Verbascum, Paulownia, Globularia, and Adonis.

9. The method, as recited in claim 6, wherein said first active ingredient berberine is extracted from one or more natural herbs selected from the group consisting of Berberis, Chelidonium, Stephniz, Coptis, Phellodendron, and Ziziphus, wherein said second active ingredient catalpol is extracted from one or more natural herbs selected from the group consisting of Rehmannia, Verbascum, Paulownia, Globularia, and Adonis.

10. A method of restoring insulin beta cells in a living object to lower a plasma sugar level, which comprises the steps of: administering a composition to the object, wherein said composition has active ingredients consisting of a first active ingredient berberine, a second active ingredient catalpol, and a third active ingredient oleanolic acid.

11. The method, as recited in claim 10, wherein said composition is manufactured into a unit dosage form having a dosage of active ingredients within a range between 1 to 300 mg/kg/dl for a single intake.

12. The method, as recited in claim 10, wherein said composition is manufactured into a unit dosage form having a dosage of active ingredients within a range between 5 to 150 mg/kg/dl for a single intake.

13. The method, as recited in claim 11, wherein said unit dosage is in a form of packet powders, solution in vials, ampoules or pre-filled syringes, capsule, tablet, draught in water, syrup or cachets.

14. The method, as recited in claim 12, wherein said unit dosage is in a form of packet powders, solution in vials, ampoules or pre-filled syringes, capsule, tablet, draught in water, syrup or cachets.

15. The method, as recited in claim 13, wherein said first active ingredient berberine is extracted from one or more natural herbs selected from the group consisting of Berberis, Chelidonium, Stephniz, Coptis, Phellodendron, and Ziziphus, wherein said second active ingredient catalpol is extracted from one or more natural herbs selected from the group consisting of Rehmannia, Verbascum, Paulownia, Globularia, and Adonis, wherein said third active ingredient oleanolic acid is extracted from one or more natural herbs selected from the group consisting of Olea, Swertia, Astrantia, Lonicera, and Beta.

16. A method of restoring insulin beta cells in a living object to lower a plasma sugar level, which comprises the steps of: administering a composition to the object, wherein said composition has active ingredients consisting of a first active ingredient berberine, a second active ingredient catalpol, a third active ingredient oleanolic acid and a fourth active ingredient taurine.

17. The method, as recited in claim 16, wherein said composition is manufactured into a unit dosage form having a dosage of active ingredients within a range between 1 to 300 mg/kg/dl for a single intake.

18. The method, as recited in claim 16, wherein said composition is manufactured into a unit dosage form having a dosage of active ingredients within a range between 5 to 150 mg/kg/dl for a single intake.

19. The method, as recited in claim 17, wherein said unit dosage is in a form of packet powders, solution in vials, ampoules or pre-filled syringes, capsule, tablet, draught in water, syrup or cachets.

20. The method, as recited in claim 18, wherein said first active ingredient berberine is extracted from one or more natural herbs selected from the group consisting of Berberis, Chelidonium, Stephniz, Coptis, Phellodendron, and Ziziphus, wherein said second active ingredient catalpol is extracted from one or more natural herbs selected from the group consisting of Rehmannia, Verbascum, Paulownia, Globularia, and Adonis, wherein said third active ingredient oleanolic acid is extracted from one or more natural herbs selected from the group consisting of Olea, Swertia, Astrantia, Lonicera, and Beta.
